(19) United States
(12) Patent Application Publication
(10) Pub. No.: US 2003/0044800 A1
(43) Pub. Date: Mar. 6, 2003

(54) Drug discovery employing calorimetric target triage

(76) Inventors: Patrick R. Connelly, Harvard, MA (US); Gregory P. Connelly, Waltham, MA (US); Janid Ali, Waltham, MA (US)

Correspondence Address: BANNER & WITCOFF, LTD., 28 STATE STREET, 28th FLOOR, BOSTON, MA, 02109, US

(21) Appl. No.: 09/946,047

(22) Filed: Sep. 4, 2001

Related U.S. Application Data
(60) Provisional application No. 60/230,548, filed on Sep. 5, 2000.
(60) Provisional application No. 60/243,496, filed on Oct. 26, 2000.

(51) Int. Cl. C12Q 1/68; G01N 33/53; G06F 19/00; G01N 33/48; G01N 33/50

(52) U.S. Cl. 435/6; 435/7.1; 702/19

(57) ABSTRACT

Novel methods for drug discovery including identification of targets and identification of the functions of targets are disclosed. The methods provide for rapid identification and high throughput screening of targets for developing therapeutics to treat disease conditions.

The present application claims priority to U.S. Patent Application No. 60/230,548 filed Sep. 5, 2000 and titled “Drug Discovery Employing Calorimetric Target Triage” and U.S. Patent Application No. 60/243,496 filed Oct. 26, 2000 and titled “Drug Discovery Employing Calorimetric Target Triage.”

### INTRODUCTION

The present invention relates to methods and processes of drug discovery that involve calorimetric detection of reactions between a target, e.g. a protein, and a member of s substrate library to determine the functions of the targets and the utilization of this data to enable drug discovery, drug development and product commercialization.

### BACKGROUND

The discovery of new pharmaceutical drugs typically has employed a procedure in which specific species, e.g. proteins, are targeted and a search for compounds that bind to those species are conducted using in vitro and/or in vivo biochemical assays. Thus, a major bottleneck in drug discovery is the identification of new targets. It is estimated that all current marketed human therapeutics are targeted to fewer than 500 proteins. During the last decade, sequencing of the human genome and genomes of important other organisms, including bacteria and fungi has greatly expanded our knowledge of the proteome, of which many novel proteins will be targets for drug discovery. It is believed that in the next 10 years, 5,000-10,000 new proteins will be available as targets for drug discovery. This suggests that the vast majority of drugable targets have yet to be found. The present invention provides efficient drug discovery methods and procedures that can be used to discover new and useful drugs.

### SUMMARY

In accordance with a first aspect, a novel method of identifying drug discovery targets is disclosed. As used here target or targets refers to proteins, lipids, carbohydrates, phospholipids, amino acids, peptides, nucleosides, nucleic acids, DNA, RNA and the like. Preferably targets refer to proteins such as membrane proteins, integral proteins, peripheral proteins, extracellular proteins, cytosolic proteins, organelle proteins, nuclear proteins, fibrous proteins, globular proteins, chaperones, and the like. Most preferably targets refer to proteins having enzymatic properties and/or transport properties. That is, most preferably targets refer to proteins capable of catalyzing one or more chemical, biological or biochemical reactions. Preferably, a multi-step procedure is used to identify one or more targets. However, one or more steps in the multi-step procedure may be omitted depending on information known about the target. Additionally, it may be necessary to add other steps depending on the information known about the targets and the results discovered using the steps outlined here. The method comprises:

(a) assaying a target by

(i) assigning a putative function to the target;

(ii) providing a library of interaction candidates;

(iii) providing a reaction mixture comprising the target;

(iv) contacting the target with the one or more members of the library of interaction candidates and measuring a value corresponding to a resultant change in heat output for at least one selected member of the library of interaction candidates;

(b) launching drug discovery employing the target comprising determining at least one pharmacological property of the at least one selected member of the library of interaction candidates.

In certain embodiments, the value corresponding to the resultant change in heat output can be compared with other predetermined values, for example. In certain preferred embodiments, the method comprises:

(a) assaying a target by

(i) assigning a putative function to the target;

(ii) providing a library of interaction candidates;

(iii) providing a reaction mixture comprising the target;

(iv) contacting the target with the one or more members of the library of interaction candidates;

(v) evaluating a change in heat output of the reaction mixture; and

(vi) optionally comparing the value for heat change obtained with a predetermined value;

(b) optionally ranking the target against other targets;

(c) launching drug discovery employing the target by determining at least one pharmacological property of the target or the one or more members of the library of interaction candidates.

In yet other embodiments, the method comprises

(a) identifying and selecting a protein target;

(b) assaying the protein target by:

(1) assigning a putative function to the protein target;

(2) providing a library of interaction candidates;

(3) providing a reaction mixture which includes the protein target;

(4) contacting the target with one or more members of the library of interaction candidates;

(5) evaluating a change in heat output of the reaction mixture;

(6) optionally comparing the value for heat change obtained with a predetermined value;

(c) launching drug discovery using at least one member of the library of interaction candidates that elicits a change in heat output.

As used here pharmacological property refers to potency, bioavailability, efficacy, dissociation constants, binding affinity, toxicity, metabolism, pharmacokinetics, pharmacodynamics, biotransformations, excretion, clearance, distribution, half-life, rate of absorption, dosage, loading dosage, maintenance dosage, mechanism of action, receptor binding, etc. As used here substrate libraries refers to a collection of compounds for which targets can be screened against for potential interaction, e.g., enzymatic activity. Typical substrate libraries may include native substrates (if known or predicted), substrate analogs, transition state analogs, potential products, substrate derivatives, and the like. Interaction, e.g., enzymatic activity will typically result in a change in heat output that may be detected by numerous methods including the methods disclosed here, such as ACTT. In many instances, the function(s), sequence, properties, etc., of the target may be unknown. The methods disclosed here are designed to identify targets, identify the functions of those targets and to identify substrate libraries and compounds that are capable of binding to the targets and inhibiting, or activating as the case may be, the targets. For example, in certain embodiments, one or more members of the substrate libraries bind to and inhibit the enzymatic activity of one or more targets, whereas in other embodiments one or more members of the substrate libraries bind to and activate one or more targets. One skilled in the art given the benefit of this disclosure will be able to select targets suitable for use in the methods described here. The method may further include identifying and selecting targets, processing the targets, and characterizing the targets, as discussed in detail below.

In accordance with another aspect, the identification and selection of targets preferably is based on information such as disease biology such that a connection or link can be established between a disease state or a medical condition and a potential target. Such information may include but is not limited to sequence information, bioinformatics information, pathology and clinical data, information resulting from genomics research, and the like. Selection of the targets preferably is based on the likelihood that the target may be associated with or involved in a certain disease state. That is, selection is based on the probability that one or more targets may be associated with a disease state. Thus, identification of one or more members of a substrate library that binds to and inhibits a target, for example, may lead to the design of therapeutics that can treat the disease state involving the target. Therefore, using the methods described here rapid identification of targets and therapeutics that bind to, and preferably inhibit the targets, can be readily identified.

In accordance with additional aspects, the targets are preferably processed prior to assaying the targets. Such processing may include but is not limited to expression of the target in a host system, e.g. bacterial system having a suitable vector containing the DNA sequence of the target, purification of the target using standard chemical and biological techniques such as chromatography, electrophoresis, and the like. Processing may also include preparation of variants, e.g. mutants, of the targets for testing and comparison to the native targets. Suitable methods for preparing target variants are well known to those skilled in the art and include but are not limited to point mutations, random mutations, and the like. One skilled in the art given the benefit of this disclosure will be able to prepare variants for use in the methods described here.

In accordance with additional aspects, the targets are preferably characterized after selection and identification and processing. Suitable characterization methods include chemical and biological methods. For example, spectroscopic techniques such as NMR, EPR, fluorescence, phosphorescence, light scattering, circular dichroism, UV-Visible absorption, infrared spectroscopy, photoacoustic spectroscopy, mass spectroscopy, calorimetry, etc. can be used to characterize the targets. Other characterization methods include enzymatic assays for identifying potential functions of the targets, physical properties for identifying the make-up of the targets, e.g. the sequence of the target, etc. In embodiments where the targets are proteins, other suitable methods such as differential scanning calorimetry can be used to provide evidence that the isolated target is in its native form, e.g. is folded properly.

In accordance with additional aspects, assaying the targets may be preferably accomplished using standard binding analyses, e.g. Scatchard analyses, competition analyses, etc. In certain preferred embodiments, Althexis Calorimetric Target Triage (ACTT), as described in below and in U.S. patent application Ser. No. 09/453,122 the entire disclosure of which is hereby incorporated by reference for all purposes, is used to assay the targets. ACTT typically provides for rapid and high-throughput screening of the targets to eliminate one or more targets which do not bind to one or more members of a substrate library. For example, one or more purified targets, e.g. purified proteins, can be placed into a vessel, e.g. a test tube, Eppendorf, vial, etc. with one or more members of a substrate library, e.g. one or more potential substrates or substrate analogs for that target. The vessel can be placed into a calorimeter, more preferably a micro-calorimeter and most preferably a high-throughput micro-calorimeter, to determine if there is enzymatic turnover of any of the substrates. This process identifies whether there is an activity associated with the target and allows for the determination of what specific substrate(s) generated the activity and subsequently identify which substrates bind to the target. Determination of which specific substrate binds to a target can be carried out by process of elimination using additional calorimetric runs with fewer than all the original substrates or other suitable means, such as Scatchard analysis using radioactively-labeled members of the substrate library, for example. This step involves assembling one more substrate libraries. Exemplary discussion of assaying the targets and the nature and types of targets and substrate libraries that can be used may be found below in the Section titled Thermo-Chemical Sensors and Uses Thereof.

In accordance with additional aspects, the targets may be further assayed using post-substrate identification analysis. In post-substrate identification analysis, the product of substrate transformation can be determined. That is, the product(s) that results from enzymatic turnover in the presence of one or more members of a substrate library can be determined. By identifying the product, the full reaction that the enzyme or protein carries out becomes evident. A high-throughput assay based on that substrate, substrate analogs, transition state analogs, products and product analogs may then be configured. Such high-throughput assays typically are enzymatic assays designed to test compound libraries for interferences with the action of that enzyme. For example, testing may involve addition of a substrate into a vessel with an enzyme and monitoring for the appearance of a product or disappearance of the substrate. Compounds that interfere with the reaction process, e.g. competitive, non-competitive, and uncompetitive inhibitors, are then tested using known drug discovery techniques and procedures to determine their suitability for a drug discovery program. Compounds are then further refined and optimized, again using known drug discovery techniques and procedures. After refinement and optimization, one or more compounds may then proceed to pre-clinical and clinical development candidates and, eventually, to a useful marketed drug. For example, one or more compounds can be administered to a mammal in a pharmaceutically effective amount, optionally with one or more binders, to treat a disease state. Such compounds may be administered according to standard dosage and administration regimens, e.g. once daily, twice daily, etc. Suitable administration amounts, e.g. 1-10 μg compound/kg of body weight, 1-100 μg compound/kg of body weight, 1-10 mg compound/kg of body weight, 10-100 mg/kg of body weight for example, can readily be determined by those skilled in the art given the benefit of this disclosure.

In accordance with additional aspects, post-substrate identification analysis can also contribute to the elucidation of the mechanism of action of the enzyme. Knowledge of the mechanism of action of an enzyme provides information relating to the transition state of the enzyme that can be used to design inhibitors based on that enzyme mechanism, e.g. non-cleavable or non-hydrolyzable transition state analogs. An additional aspect of post-substrate identification analysis is an investigation into the three-dimensional structure of that protein. Structure can be determined, for example, through x-ray crystallography, or high-field nuclear magnetic resonance spectroscopy in conjunction with molecular modeling studies. A detailed knowledge of the site on the protein where the substrate binds, e.g. the active site, can provide insight into the chemical structure of potential inhibitors, which often make use of specific interactions with nearby atoms of the protein. Thus post-substrate identification analysis provides at least three avenues one can use to optimize small molecules as drugs: 1) it can form the basis of a configurable high-throughput assay for drug discovery lead identification, 2) it can provide information about an enzyme's mechanism, which is classically the way that enzyme inhibitors as drugs are designed, and 3) owing to the high solubilities of substrates in general (relative to inhibitors), one can then readily solve the structures of the targets with a substrate or substrate-analog bound, providing insight into the nature of potential pharmacophores and intermolecular interactions.

In accordance with additional aspects, the targets can optionally be ranked. Ranking of targets can aid in the selection of proteins as targets for drug discovery and typically involves a number of criteria. For example, the targets can be ranked such that targets with the highest rankings may be selected for drug discovery. Such rankings may include but are not limited to type of reaction catalyzed, binding affinity for one or more members of a substrate library, enzymatic turnover number, solubility, molecular weight, localization of the target, e.g. extracellular, cytosolic, organelle, and the like. Targets may be ranked according to the association with a disease state, for example, the relatedness of the target and the disease state of interest. Studies to determine this relatedness may involve essentiality studies using gene knockout technology in bacteria, fungi, animal cells or human cells. These studies can show that a gene and the gene products are essential if, when removed from a cell, the cell fails to survive. Other criteria may include the suitability of the chemical structure of the protein and the likelihood of developing a small molecule inhibitor of the protein. In some cases knowledge of the structure and function of the protein will provide leads to the type of compounds that may be useful inhibitors of the protein. For this information the tractability of chemical synthesis, the suitability for pharmaceutical use, and the commercial viability of a program can be estimated. One skilled in the art given the benefit of this disclosure will be able to rank and select targets and substrate libraries suitable for drug discovery.

In accordance with additional aspects, certain targets are chosen for drug discovery. Target selection typically is based on knowledge of disease biology, computational chemistry, bioinformatics, mechanistic enzymology, potential chemistry based on substrate and target characteristics, and structural information for the particular target. Preferably targets are chosen that bind to one or more members of a substrate library. In accordance with other aspects, drug discovery is launched using the chosen targets. The process, preferably in accordance with well-known drug discovery protocols and methods, involves identification of assay “hits,” developing them into “leads,” proceeding to compound optimization and finally initiation of pre-clinical and clinical development. Such pre-clinical and clinical testing methods are well known to those skilled in the art and involve in vitro, in vivo, and animal testing, e.g. testing in mammals such as rats or humans, as well as other standard FDA approved procedures for clinical testing of potential therapeutics.

It is a significant advantage that preferred embodiments of the novel multi-step drug discovery method disclosed and described here is scalable. That is, the method described here are scalable to meet the current demands of the pharmaceutical industry. The methods can be further scaled to meet future pharmaceutical industry needs such that thousands of targets can be handled in a commercial time frame and with practical resources.

### BRIEF DESCRIPTION OF DRAWINGS

Certain preferred embodiments of the invention will be described below with reference to the attached drawings in which:

FIG. 1 is a representation of identifying and selecting potential targets;

FIG. 2 is a representation of processing of selected targets;

FIG. 3 is a representation of characterizing the targets;

FIG. 4 is a first representation of assaying the targets using ACTT;

FIG. 5 is a second representation of assaying the targets using ACTT;

FIG. 6 is a representation of selecting targets for drug discovery;

FIG. 7 is a representation of launching drug discovery;

FIG. 8 depicts a schematic diagram of the target-mediated conversion of a test substrate(s) into a product(s). Such conversion generates a heat signal. This method measures the heat output generated from the interaction between a test substrate and a target (e.g., a target protein). As shown, the target-mediated conversion of a test substrate(s) into a product(s) generates a heat signal. The heat signal can be detected calorimetrically;

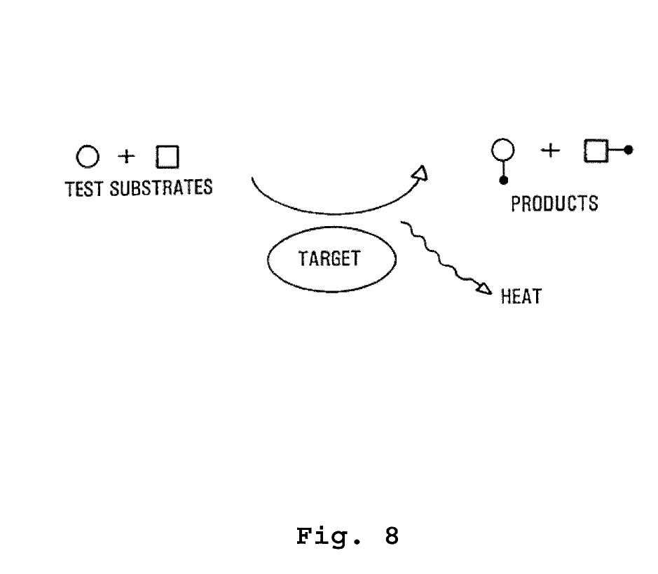

FIG. 9 depicts a schematic diagram of the molecular detection switch to detect binding of a test ligand. This method uses the generation of a heat signal to identify the interaction of a test ligand with a target (e.g., a target protein). As shown, a surrogate ligand is incubated in the presence of the target such that an interaction (e.g., binding) occurs. Upon binding of the test ligand, the surrogate ligand is displaced. The free surrogate ligand (i.e., the displaced surrogate ligand) serves as a substrate for a signal-generating entity, e.g., an enzyme, in such a manner that a heat signal is generated. An important feature of the signal-generating entity is that it produced a large amount of heat per unit time (i.e., it amplifies the binding signal). The heat signal can be detected calorimetrically;

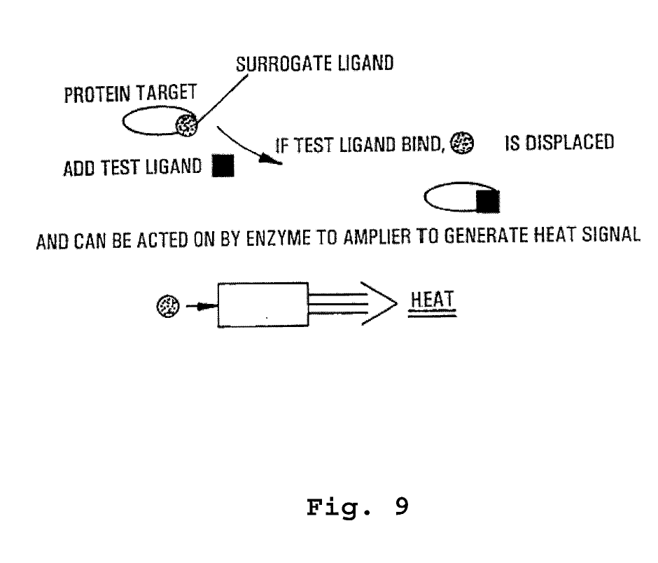

FIGS. 10A-10B depict a read-out of the rate of heat generated (μcal/sec) during a substrate screen with hexokinase with respect to time (sec).

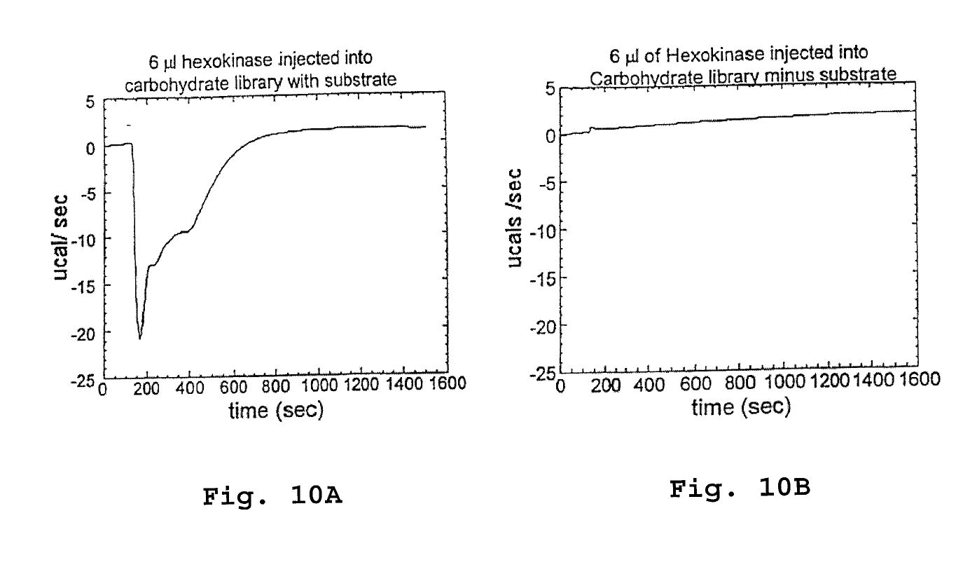

FIG. 10A shows the heat flow in the presence of a substrate, which reaches a maximum soon after the addition of enzyme to the reaction cell and then decays to the baseline as the level of substrate is depleted.

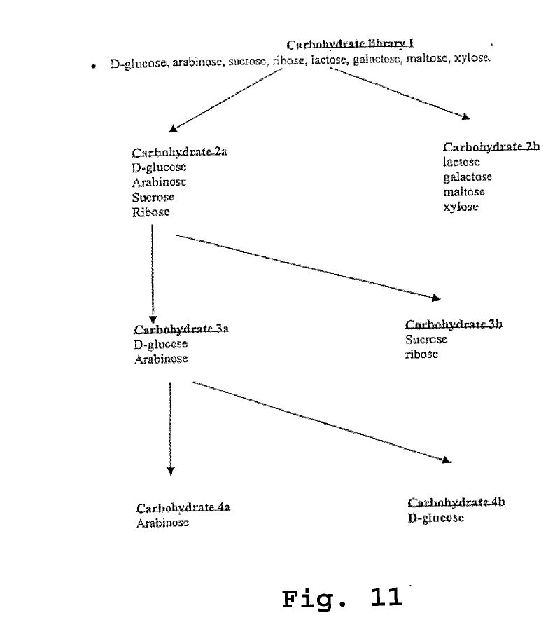

FIG. 10B shows a control (carbohydrate library minus substrate);

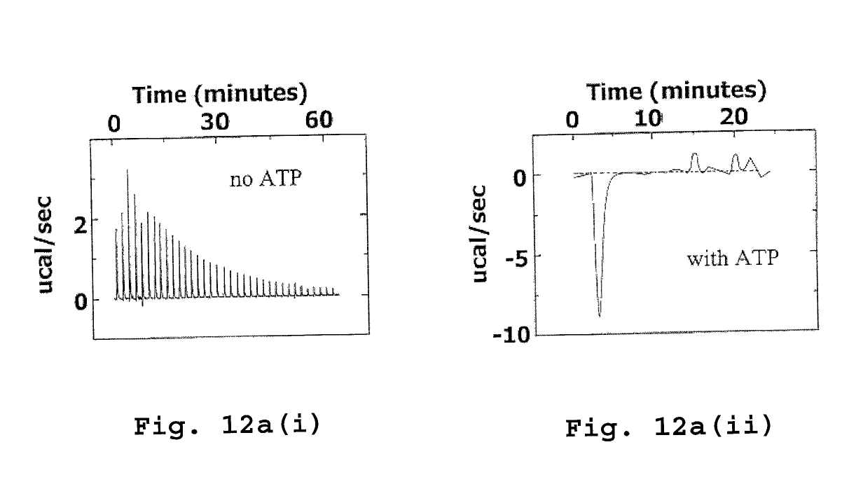

FIG. 11 depicts the experimental flow chart for the substrate screen with hexokinase;

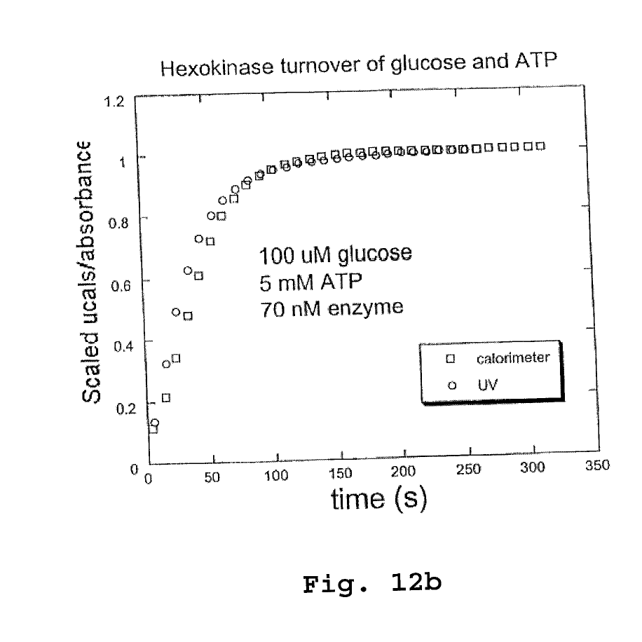

FIG. 12a(i) shows the heat released upon binding of hexokinase to glucose over time. As time proceeds, the fixed amount of hexokinase in the cell is bound, so additional glucose produces less heat. 5 μL injections of a 5 μM solution of glucose were titrated into the calorimetric cell containing 2 mL of a 50 nM solution of hexokinase. Because no ATP is present, the glucose merely binds to the protein. The heat released from each injection was measured and the binding constant calculated from those measurements;

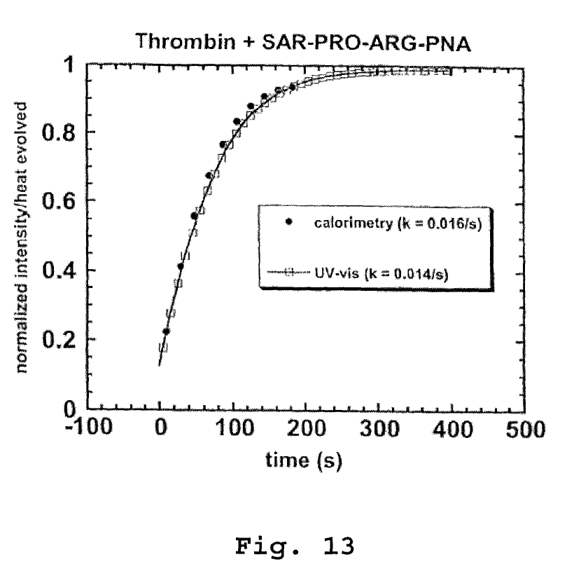

FIG. 12a(ii) shows heat released as hexokinase phosphorylates glucose in the presence of ATP converting ATP to ADP. A single 5 μL injection of a 20 μM solution of hexokinase was titrated into the calorimetric cell containing 2 ml of a solution of 100 μM glucose and 1 mM ATP. A large negative heat is observed as the hexokinase acts on the glucose. We integrated the heat evolved with respect to time to follow the time course of the reaction;

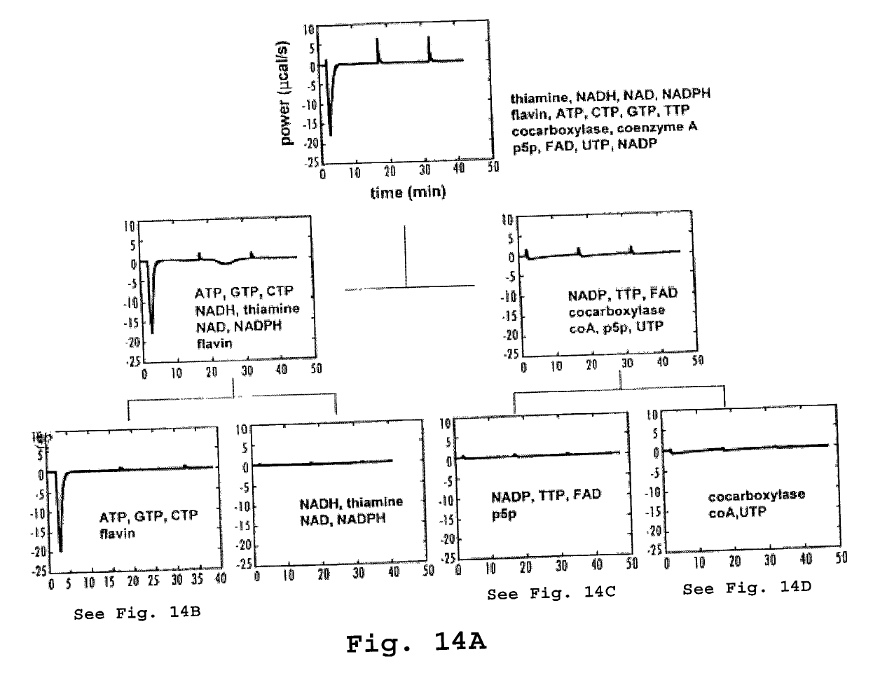

FIG. 12b compares rates of reaction of glucose and ATP catalyzed by hexokinase measured by two different techniques: UV spectrophotometry and calorimetry. The heat output of the reaction from FIG. 12a(ii) is plotted with respect to time. The reaction was also measured using a second assay, both methods give the same rates for the reaction (within experimental error);

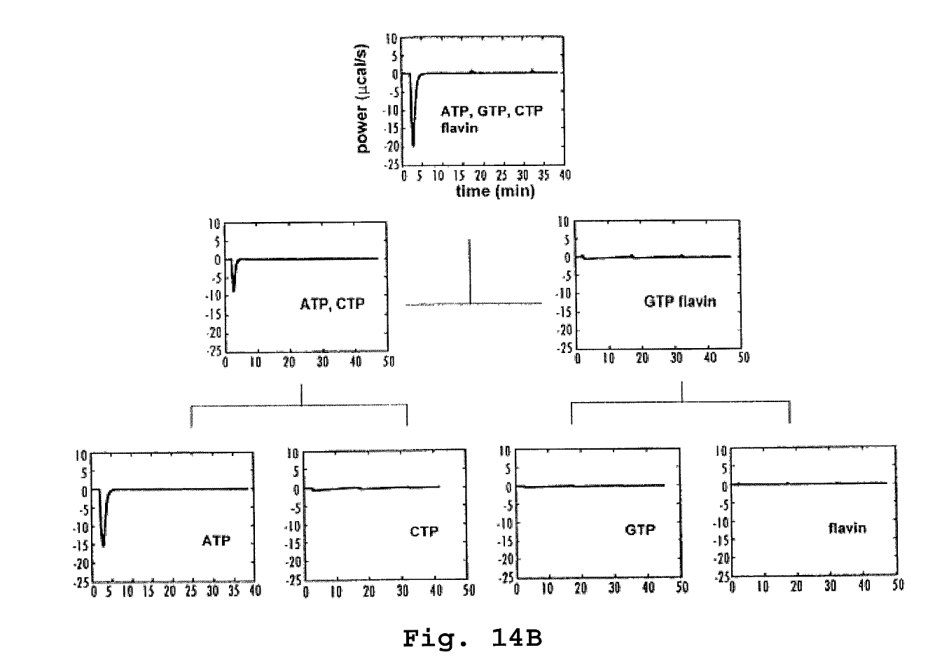

FIG. 13 compares rates of reaction of thrombin-catalyzed cleavage of a labeled peptide substrate (SAR-PRO-ARG-parantroanilide) with UV spectrophotometry and calorimetry. Upon cleavage, the PNA (paranitronalide) label absorbs more UV radiation and heat is evolved. 2 ml of a solution with 186 nM thrombin and 250 μM substrate were allowed to react, and the reaction was monitored calorimetrically and spectrophotometrically. Again, nearly identical rates were calculated using the different assays;

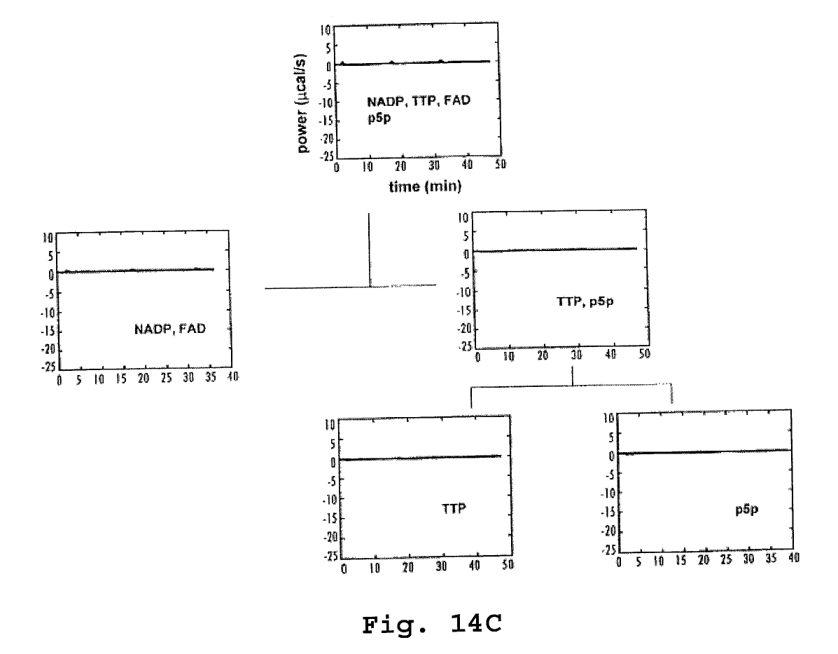

FIGS. 14A-14D demonstrate the use of the present invention to deconvolute a mixture of compounds. Hexokinase and glucose were present in each test. Various mixtures of cofactors were added to each test. Only when ATP was present in the mixture was a significant amount of heat generated. 5 μL injections of a 20 μM solution of hexokinase were titrated into a solution of 100 μM glucose and one or more “cofactors”, all at 1 mM concentration. In the initial experiment with the entire library of 15 cofactors present, enzymatic turnover was observed (as in FIG. 12a(ii)). We were then able to successfully separate out the cofactors in subsequent experiments to determine which one was actually necessary to allow the hexokinase to turn over the glucose. A large negative heat is observed in the first injection of protein whenever ATP, the correct cofactor, is present in the mix of cofactors in the cell. Only tiny heats of dilution are observed when ATP is absent;

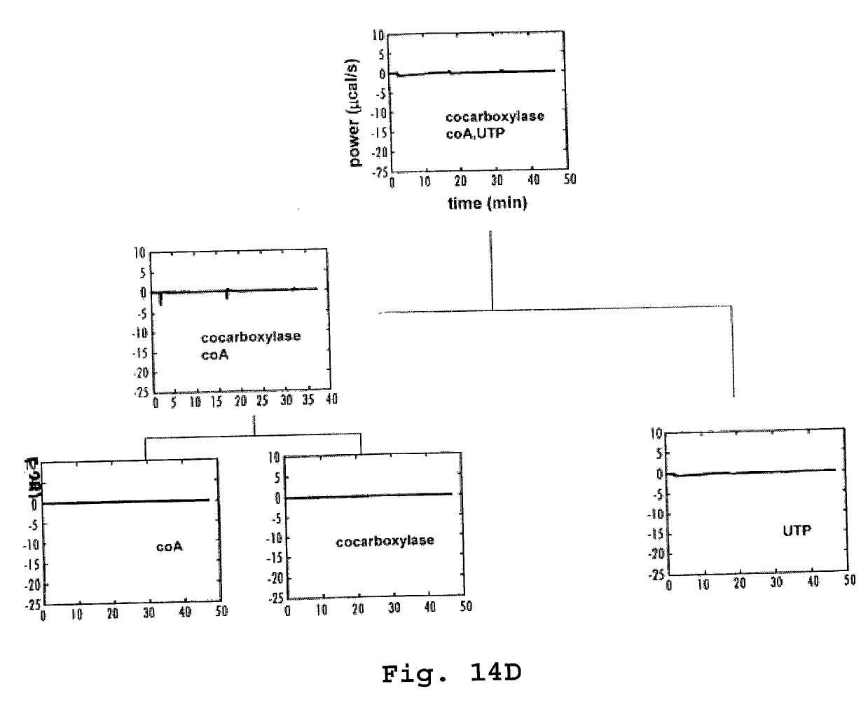

FIG. 15 demonstrates calorimetric measurement of hexokinase-catalyzed glucose phosphorylation in the presence of a complex mixture of natural products. 5 μL injections of a 20 μM solution of hexokinase were titrated into solutions of 100 μM glucose and 1 mM ATP and increasing concentrations of tea. Tea solutions can simulate natural product extracts, i.e., complex mixtures. Significant enzymatic activity (turnover) was observed at all but the highest concentrations of tea; and

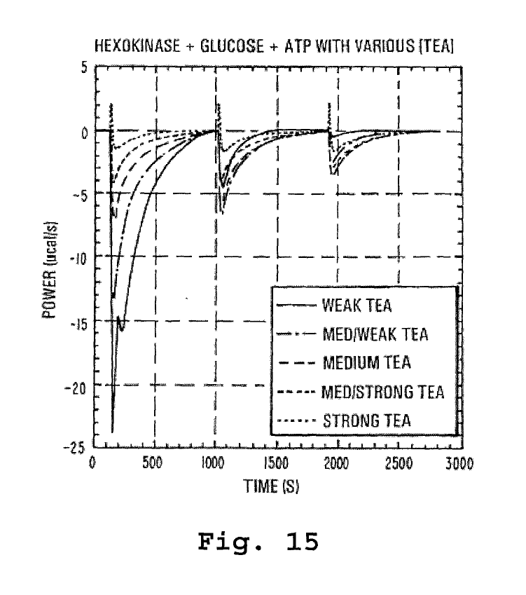

FIG. 16 demonstrates the use of calorimetry to aid in determining function of cryptic proteins. E. coli protein YJEQ binds to GTP analog GTP-gamma-S. A K_{D} of 115 μM was determined. 5 μL injections of an 11 mM GTP-gamma-S solution were titrated into 2 mL of a solution containing 385 μM of an E. coli protein for which no function was previously known. As in FIG. 12a(i), the experiment shows that the protein binds this molecule (an analog of GTP), allowing us to putatively assign GTP-binding properties to this protein.

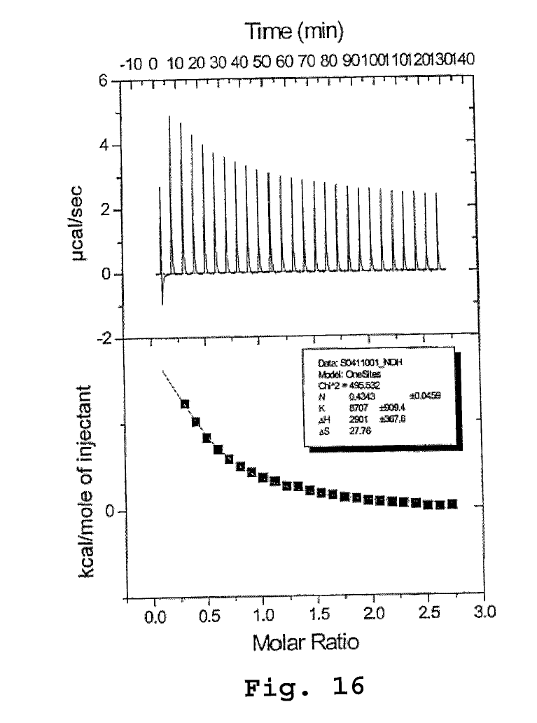

### DETAILED DESCRIPTION OF CERTAIN PREFERRED EMBODIMENTS

I. Drug Discovery

In accordance with certain preferred embodiments, targets can be identified and selected using numerous methodologies. Preferably the targets are identified and selected by associating potential targets with one or more disease conditions. Such associations typically are elucidated using clinical and laboratory research. For example, disease conditions whose symptoms are caused by a defective gene may be linked to the absence of as suitable protein or the presence of a defective protein, for example. That is, the defective gene may produce a protein whose activity is reduced to a level such that normal cellular function is not sustained, e.g. the protein is essential to normal function of the cell, or the defective gene may produce a protein which alters the cellular properties of the cell, e.g. the defective protein may produce unwanted side effects such as initiation of transcription of one or more genes. Thus activation and/or inhibition of the protein would be one potential avenue to treat the disease state. Therefore, the native protein can be selected as well as the defective protein for identifying members of substrate libraries that bind to each of the proteins. Such comparisons provide insights into the role of a protein in cell function.

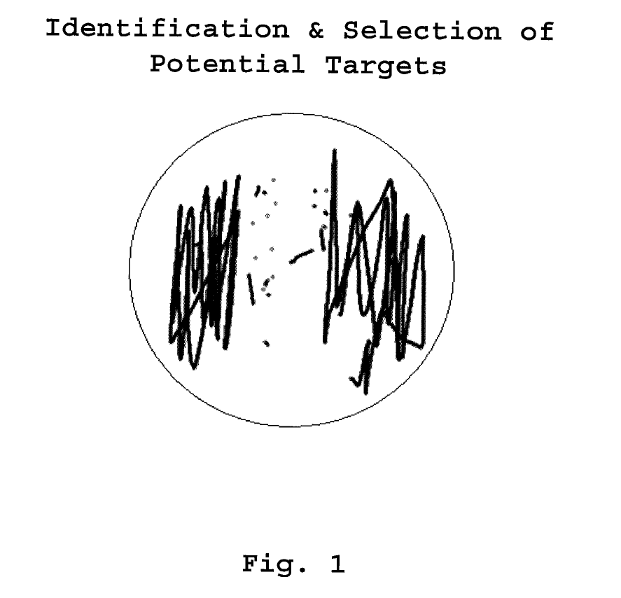

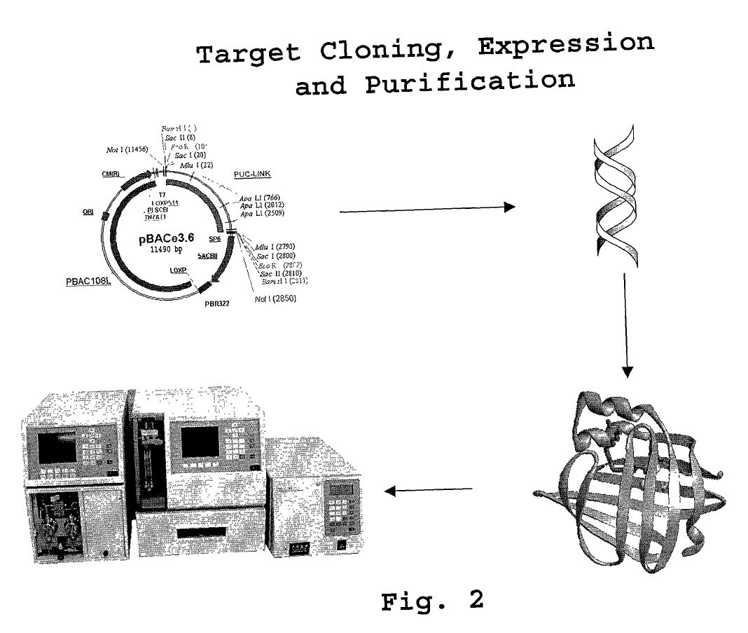

In accordance with preferred embodiments, referring to FIGS. 1 and 2, targets may be identified and selected using well-known chemical, biological, and molecular biological techniques. For example, defective genes that are linked to a disease condition can be cloned using suitable expression systems, e.g. bacterial expression systems, yeast expression systems, mammalian cell expressions systems, and the like. The products of the cloned gene, e.g. RNA and protein for example, can be produced in large amounts by choosing an expression system that is designed to over-express protein in the presence of certain compounds, e.g. lactose in the case of an inducible vector containing the lac operon. After expression of the targets, the targets can be isolated using chromatographic and electrophoretic techniques, e.g. affinity chromatography, column chromatography, HPLC, LC, capillary electrophoresis, gel electrophoresis, etc. Any selection tags present, e.g. glutathione-S-transferase tags, histidine tags, and the like, may be removed from the expressed protein prior to testing using well-known techniques known to those skilled in the art for removal of expression tags. One or more purified targets can be tested alone against one or more members of a substrate library, or purified targets can be pooled for testing against one or more members of a substrate library, as discussed in detail below.

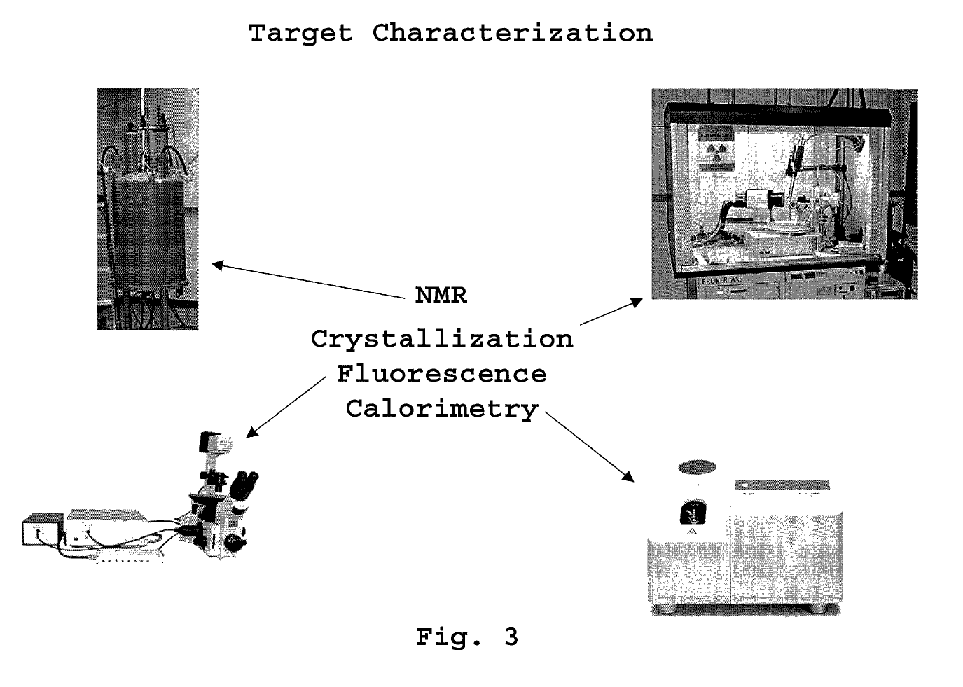

In accordance with certain preferred embodiments, referring to FIG. 3 the purified targets are typically characterized prior to assaying the targets. That is, some of the physical, chemical, and/or physicochemical properties of the targets are elucidated prior to testing the targets against members of the substrate libraries. Such testing typically provides some indication of the physical structure and properties, e.g. two- or three-dimensional structure, of the targets as well as potential functions of the targets, e.g. potential enzymatic function. Target characterization typically is performed to identify likely members of a substrate library that might bind to the target. That is, based on target characterization, one or more members of a substrate library can be selected that have a high probability of binding to the targets. Additionally, one or more members of the substrate library that are not likely to bind to the targets can be selected as a control. Such methods decrease the time required to identify substrates, and thus potential therapeutics, that can bind to the targets. In other embodiments, the substrate libraries are chosen randomly. Random choosing of substrate libraries may be especially suitable when little or no information can be obtained about the target's properties or function. One skilled in the art given the benefit of this disclosure will be able to select suitable techniques for characterizing the targets. Exemplary techniques include but are not limited to NMR, EPR, circular dichroism, light scattering, UV-Visible absorption, infrared spectroscopy, enzymatic assays, fluorescence, phosphorescence, photoacoustic spectroscopy, mass spectroscopy, and the like.

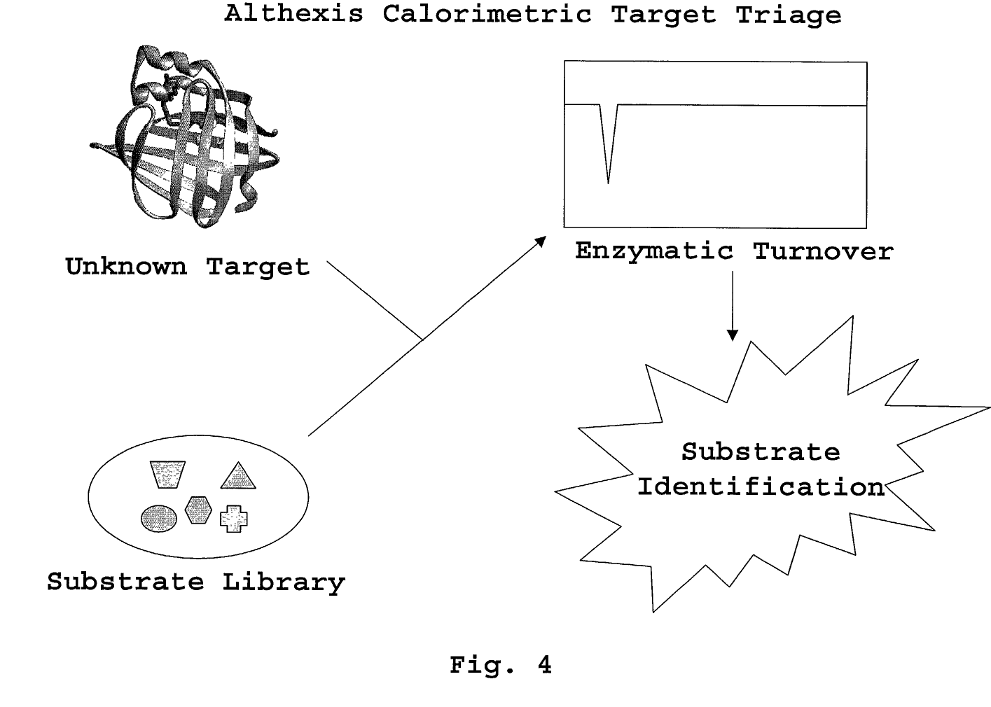

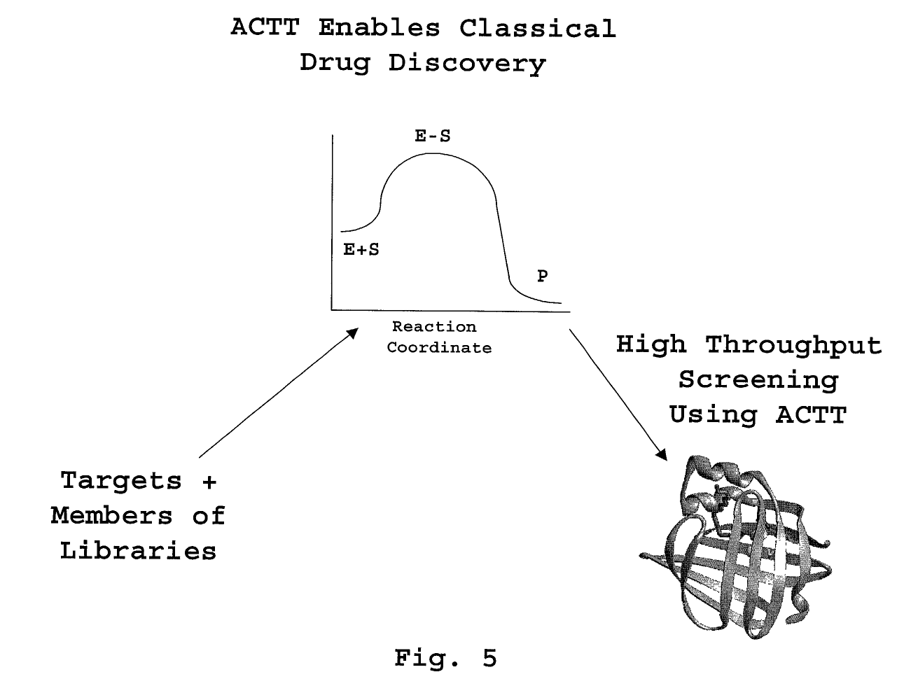

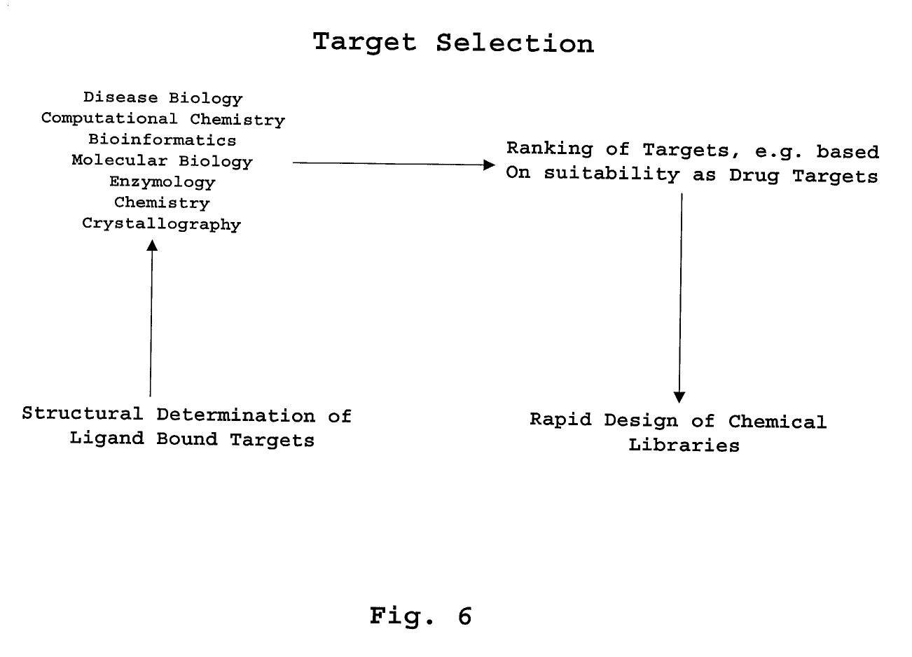

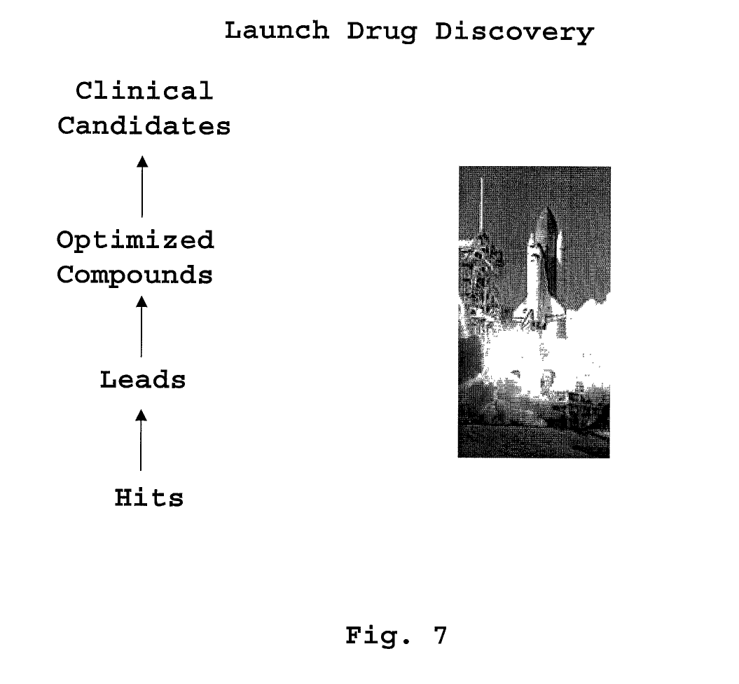

In accordance with certain preferred embodiments, referring to FIGS. 4-7, the targets can be assayed using members of substrate libraries to identify one or more compounds that bind to one or more targets. Such assays can be conducted using standard binding methodologies well known to those skilled in the art. Preferably such target assays are conducted using calorimetry, such as ACTT, which is described in detail below. In preferred embodiments, a change in temperature is used to monitor for binding of one or more substrates to one or more targets. The change in temperature may be monitored directly, e.g. using a thermometer, thermocouple, and the like, or may be monitored using calorimetry. Preferably the members of the substrate libraries are tested batchwise. That is, a sample comprising 10, 20, 30 or more members of a substrate library are tested with one or more targets. When no heat is evolved (exothermic reaction) or required (endothermic reaction), all substrates in that sample can be eliminated as potential substrates for the target. When heat is evolved or required, then the sample can be subdivided for further testing. For example, if a sample containing 10 members of a substrate library and a single target produces heat, then the substrate library members in that sample may be subdivided into 2 samples containing 5 members each. The procedure can be performed again using the target. That is, each of the 2 samples containing five members can be tested against the target. Any sample producing no heat or requiring no heat can be discarded while samples producing heat are further subdivided, e.g. tested individually, for example, to identify which members of the substrate library bind to the target. One skilled in the art given the benefit of this disclosure will be able to select suitable testing methods for rapidly determining members of a substrate library that bind to one or more targets.

In accordance with certain preferred embodiments, the products of an enzymatic reaction using the substrate libraries and the targets can be identified. That is the products can be isolated using well known chemical techniques, e.g. dialysis, filtration, chromatography, and the like, and can be characterized and identified using well known chemical techniques, such as NMR, HPLC, mass spectroscopy, etc. By comparison of the products and the substrates, information about the active site geometry and enzyme function can be obtained. Such information may include but is not limited to the structure of the transition state, e.g. the transition state geometry and the type of reaction, e.g. addition, oxidation, reduction, etc. This information may be useful in the design and synthesis of inhibitors, substrate analogs, transition state analogs, etc. One skilled in the art given the benefit of this disclosure will be able to select suitable methods for determining active site geometries and enzyme function.

In accordance with certain preferred embodiments, drug discovery may be launched based on members of substrate libraries that bind to, inhibit, or activate one or more targets. Chemical libraries that contain compounds similar to the library members that bind to the target may be synthesized. For example, derivatives, analogs, homologs, etc. can be synthesized and tested for binding to a target using, for example, ACTT. Preferably, compounds that have a lower dissociation constant than the native substrate of a target are chosen for drug discovery. Preferably compounds having a dissociation constant that is five-times, ten-times, twenty-times, fifty-times, or 100-times lower than the dissociation constant of the native substrate is chosen for launching drug discovery. Drug discovery procedures include in vivo testing, in vitro testing, animal testing, testing in humans, preclinical trials, clinical trials, etc. One skilled in the art given the benefit of this disclosure will be able to select suitable drug discovery procedures for developing therapeutics to treat a disease state.

II. Thermochemical Sensors and Uses Thereof

The following section discusses thermo-chemical sensors and their uses, which are applicable to the methods disclosed here and principally for the practice of at least the fourth step, step (d) in the Summary, of the multi-step process disclosed here.

A. Summary of Thermochemical Sensors

Methods described herein link the interaction, e.g., binding, of a test compound, e.g., a test ligand or a test substrate, with a target (e.g., a target protein or nucleic acid) to a change in heat. The heat output is detected by calorimetry. This allows analysis of the interaction without imposing sharply constraining limitations on the type, range, or specific identity of the activity of the target. By way of example, it allows for the identification of an interactor, e.g., a substrate, for a target having an unknown, poorly characterized, or merely putative or broadly described activity. e.g., where the target is an enzyme, methods of the invention detect a change in heat generated upon conversion of a test substrate(s) into a product(s) or, where the target and interactor are ligand and counter-ligand, upon binding. The absorption or evolution of heat is a universal property of chemical reactions, thus the power of the methods of the invention can transcend that of methods which make overly constraining limiting assumptions about the nature of the target or its nteractions with other molecules. Some embodiments of the invention require no assumptions about the nature of the target and its interaction with its interactor, e.g., its naturally occurring ligand, substrate, or binding partner. Other methods of the invention incorporate knowledge of or assumptions about the target (and/or interactor) to guide in the choice of potential interactors. e.g., embodiments of the invention use genomic, or other bioinformatic analyses of the target to optimize and prioritize the choice of interactors against which to test the target.

Accordingly, in one aspect, the invention features, a method of analyzing a target, e.g., a protein. (Although the method is described with regard to a protein, other target molecules, e.g., other macromolecules, e.g., nucleic acids, can be analyzed with the methods described herein.)

The method includes:

(1) optionally, assigning a putative function to the target, e.g., protein. Putative function can be assigned by any means, e.g., by the identification of a characteristic possessed (or in some cases not possessed) by the target. Exemplary characteristics include: a structural characteristic, e.g., in the case of a protein, a preselected level of sequence identity with another protein; possession of a sequence or motif, or a protein fold; similarities in 3-dimensional structure between the target and another molecule, e.g., a protein of known function; promoter structure or other 3′, 5′, or other regulatory structure; chromosomal location or other genetic properties such as suppressor, auxotroph, permease, drug resistance, drug sensitivity, or other similar activities or properties; expression profile, e.g., tissue specificity, disease, or disorder specific expression, temporal expression pattern; source, e.g., the species from which the target is derived. The identification of a characteristic shared (or in some cases not shared) by the target and a molecule, e.g., a protein, of known function can allow assignment of the, or an, activity of the molecule, e.g., protein, of known function to the target. For example, putative function can be assigned, e.g., by comparing the sequence of the protein, or a nucleic acid which encodes it to a reference sequence, (e.g., determining if the target protein includes a preselected sequence, e.g., a preselected motif, e.g., a consensus sequence), or by comparing the sequence of the protein to another protein with known 3-dimensional structure (e.g., determining the presence or absence of a protein fold); or by comparing the 3-dimensional structure of a crystal of the target protein to other proteins of known function;

(2) providing a library of interaction candidates, e.g., a library of potential substrates, or binding ligands, for a protein. (Preferably the library will include at least one, and more preferably a plurality of members, each of which is known to interact with a protein having the assigned putative function. By way of example, a library can contain a plurality of protease substrates. Assignment of putative function can optimize screening strategy, e.g., by guiding the choice of a particular library of interactors.);

(3) providing a reaction mixture which includes the target, e.g., protein:

(4) contacting the target, e.g., protein, with a member of the library,

(5) evaluating a change in heat output of the reaction mixture;

(6) optionally, comparing the value for heat change obtained with a predetermined value, thereby analyzing the target, e.g., protein, e.g., by identifying a library member which is a substrate or a ligand of the target, e.g., protein. (In this method, as well as with all other methods described herein the assignment of letters or numbers to the steps of a method is merely for the convenience of the reader and, unless otherwise required, does not mean that the steps must be performed in the recited order.)

In a preferred embodiment, the target, e.g., a protein, is produced by a pathogen, e.g., a prokaryotic or a eukaryotic pathogen, including a bacterium, a protozoan, a virus, e.g., phage, or a fungus. For example, the protein can be a protein produced by any of the following species: Aquifex aeolicus, Pyrococcus horikoshii, Bacillus subtilis, Treponema pallidum, Borrelia burgdorferi, Helicobacter pylori, Archaeoglobus fulgidus, Methanobacterium thermo., Escherichia coli, Mycoplasma pneumoniae, Synechocystis sp., Methanococcus jannaschii, Saccharomyces cerevisiae, Mycoplasma genitalium, Haemophilus influenzae, Rickettsia prowazekii, Pyrococcus abyssii, Bacillus sp., Pseudomonas aeruginosa, Ureaplasma urealyticum, Pyrobaculum aerophilum, Pyrococcus furiosus, Mycobacterium tuberculosis, Mycobacterium tuberculosis, Neisseria gonorrhea, Neisseria meningiditis, Streptococcus pyogenes, Borellia burgdorferi, Caulobacter crescentus, Chlorobium tepidum, Deinococcus radiodurans, Enterococcus faecalis, Legionella pneumophila, Mycobacterium avium, Mycobacterium tuberculosis, Methanococcus jannaschii, Neisseria meningitides, Pseudomonas putida, Porphyromonas gingivalis, Salmonella typhimurium, Shewanella putrefaciens, Streptococcus pneumoniae, Vibrio cholerae, Clostridium acetobutylicum, Campylobacter jejuni, Halobacterium salinarium Institute, Listeria monocytogenes, Mycobacterium tuberculosis Sanger, Mycoplasma mycoides, Neisseria meningitidis strain, Streptomyces coelicolor, Actinobacillus actinomyce, Chlamydia trachomatis, Halobacterium sp., Mycoplasma capricolum, Neisseria gonorrhea, Pseudomonas aeruginosa, Aspergillus nidulans, Candida albicans, Leishmania major, Neurospora crassa, Pneumocystis carinii, Plasmodium falciparum, Saccharomyces cerevisiae, Schizosaccharomyces pombe, Trypanosoma cruzi, Trypanosoma brucei, Abelson murine leukemia virus, Adeno-associated virus 2 or -3, Dengue virus type 1, 2 or 3, Hepatitis A-G virus, Hepatitis GB virus B, Human T-cell lymphotropic virus type 1 or 2, Human T-cell lymphotropic virus type I, Human adenovirus type 12 or 2, Human herpesvirus 1-4, Human immunodeficiency virus type 1-2, Human parainfluenza virus 3, Human respiratory syncytial virus, Infectious hematopoietic necrosis virus, Influenza A virus, Influenza B virus, Influenza C virus and Measles virus. Additional examples of species that produce the targets tested using the methods of the invention are described below.

In a preferred embodiment, the target, e.g., a protein, is produced by a eukaryotic organism, e.g., a single-celled or a multicellular organism. Examples of such eukaryotic organisms include: Arabidopsis thaliana M, Brugia malayi, Caenorhabditis elegans, Drosophila melanogaster, Shistosoma mansoni, Shistosoma japonicum, and mammals, e.g., humans. Preferably, the target is produced by a human.

In a preferred embodiment, the target, e.g., a protein, is produced by an organelle, e.g., the mitochondria, of an organism.

In a preferred embodiment, the target, e.g., the protein, has no known activity (e.g., enzymatic activity), or has an activity which is difficult to measure. In preferred embodiments, the protein has a known first activity and it is tested against a library which includes an interactor which interacts with the protein by way of a second activity, e.g., an unknown activity.

In a preferred embodiment, the target is a naturally occurring protein or fragment thereof, a protein of unknown function and/or structure; a protein for which the ligand, substrate, or other interacting molecule is not known. In other embodiments, the protein has at least one enzymatic activity.

In a preferred embodiment, the target is a nucleic acid, e.g., a DNA or RNA (e.g., structured RNA, e.g., a ribozyme).

In a preferred embodiment, a plurality of library members is tested simultaneously, e.g., in the same reaction mixture, which can allow for an increase in the throughput of the method. A plurality of library members, e.g., one which provides a positive result, can be subdivided into smaller groups and those smaller groups tested. One or more library members from the plurality or from a smaller group, e.g., one which provides a positive result, can be tested individually.

In a preferred embodiment, the method further includes repeating one or more steps, e.g., one or both of steps (4) and (5), under a different condition, e.g., at a different salt concentration, different pH, or in the presence of a different cofactor.

In a preferred embodiment, the method further includes repeating at least one step, e.g., steps (3)-(6) with a second or subsequent member or members of the library. In a preferred embodiment, a plurality of library members, e.g., candidate substrates or test ligands, is tested. In a preferred embodiment, the plurality of library members includes at least 10, 10², 10³, 10⁴, 10⁵, 10⁶, 10⁷, or 10⁸ compounds. In a preferred embodiment includes at least 10, 10², 10³, 10⁴, 10⁵, 10⁶, 10⁷ or 10⁸ of the library members share a structural or functional characteristic.

In a preferred embodiment, the library includes a plurality of members having a common characteristic, e.g., all members of the plurality are enzyme cofactors; substrates for, e.g., biosynthetic or degradative enzymes (e.g., protease substrates), including carbohydrates, nucleoside/nucleotides, amino acids, lipids; vitamins; hormones; nucleic acids; e.g., DNA molecules; or natural products, e.g., bacterial natural products. The library can include any metabolite, precursor, or intermediate of the members listed above.

In a preferred embodiment, the library is: a substrate library; a cofactor library; a carbohydrate biosynthesis and/or degradation library; a purine and pyrimidine biosynthesis and/or degradation library; an amino acid biosynthesis and/or degradation library; a lipid biosynthesis and/or degradation library; a vitamin and/or hormone library; a nucleic acid, e.g., DNA, library; or a natural product library, e.g., a bacterial natural product library.

In a preferred embodiment, a library member (a potential or candidate interactor) is a species which has potential to interact with a target, e.g., a target protein. Preferably, a library member is a candidate substrate or a test ligand.

In a preferred embodiment, a library member is selected from the group consisting of: an enzyme substrate, a metabolite, a cofactor, a natural product (e.g., a bacterial natural product), a carbohydrate, a polysaccharide, a nucleic acid (e.g., a nucleoside or nucleotide precursor, a double-stranded (ds) or single-stranded (ss) DNA molecule, a circular nucleic acid, a super-coiled nucleic acid), an amino acid, (e.g., a D- or L-amino acid or a precursor thereof), a vitamin, a hormone, a lipid, a small organic molecule, a metals, a peptide, a protein, a lipid, a glycoprotein, a glycolipid, a transition state analog and combinations thereof.

In a preferred embodiment, the method further includes testing the protein against at least one member of a second library.

In a preferred embodiment, two, or more, libraries are tested simultaneously. By way of example, the target can be tested against each (or some) members of a first library, e.g., a cofactor library, and each (or some) members of a second library, e.g., a library of potential substrates. Thus, in the case of two libraries with a first library having 50 members (first₁, first₂, . . . first₅₀) and a second library having 50 members (second₁, second₂, . . . second₅₀ . . . ) the target is tested against all or a plurality of the novel combinations, e.g., against (first₁, second₁,), (first₁, second₂) . . . (first₁, second₅₀), and so on.

In a preferred embodiment, a library member is a member of a combinatorial library.

In a preferred embodiment, the target interacts with, e.g., binds, and preferably modifies, the test compound. Modify, as used herein, includes making or breaking a bond, e.g., a non-covalent or covalent bond, in the test compound or the target. Modification includes cleavage, degradation, hydrolysis, a change in the level of phosphorylation labeling, ligation, synthesis, and similar reactions. Modification can include changes in activity, e.g., enzymatic activity, physical changes in phase, changes in aggregation, or polymerization.

In a preferred embodiment, the method further includes:

analyzing the target structure or function, e.g., analyzing the physical properties of the target; analyzing the target in vitro or in vivo activity; analyzing the target sequence (e.g., amino acid or nucleotide sequence) for the presence of, e.g., conserved amino acid domains, thereby predicting the target structure or function. In a preferred embodiment, the analysis of the target structure or function is performed prior to contacting the target with the library.

In a preferred embodiment, the method further includes:

selecting a library member, e.g., candidate substrate or test ligand based on its interaction with the target; and confirming that the candidate substrate or test ligand is a substrate or a ligand, is respectively.

In a preferred embodiment, the method further includes selecting a library member based on its interaction with the target and contacting the library member with a cell, e.g. a cultured cell, or an animal, and, optionally, determining if the library member has an effect on the cell of animal.

In a preferred embodiment, the method further includes selecting an interactor (e.g., a library member) on the basis of its interaction with the target and: purifying the library, e.g., a candidate substrate or test ligand; crystallizing a library member, e.g., a candidate substrate or test ligand; evaluating a physical property of a library member, e.g., a candidate substrate or test ligand, e.g., molecular weight, isoelectric point, sequence (where relevant), or crystal structure. The library member can be crystallized by itself, or as complexed with the target.

In a preferred embodiment, the method further includes using a library member selected for interacting with the target to identify, e.g., by binding to or interacting with the selected library member, an agent which modulates an interaction between the target and the selected library member.

In a preferred embodiment, the method further includes selecting an interactor (e.g., a library member) on the basis of its interaction with the target and: optimizing a property of a chosen library member, e.g., candidate substrate or test ligand, e.g., optimizing affinity for the target, altering molecular weight, e.g., decreasing molecular weight, or altering, e.g., increasing, solubility. Optimization can be performed using known methods or methods disclosed herein.

In a preferred embodiment, the change in heat output is measured with a microcalorimeter.

In a preferred embodiment, the method further includes determining a physical constant of an interaction between the protein and a member of the library, e.g., k_{cat}, K_{M}, or K_{D}.

In a preferred embodiment, the method can include the use of a linking reaction, e.g., a surrogate ligand, as described elsewhere herein.

In another aspect, the invention features, a method of purifying or isolating an interactor (or a target) from a mixture. (Although in the embodiment described below, an interactor, e.g. a substrate or counter ligand, is purified or isolated using the target as an assay reagent analogous methods, which isolate or purify a target using an interactor, e.g., a substrate, as an assay reagent are also within the invention.) The interactor can be, e.g., a ligand, receptor, counter ligand, cofactor, or substrate, which interacts with a target, e.g., a protein. The mixture can be a complex biological sample, e.g., whole cells, a cell homogenate or lysate, a tissue sample, a sample of a biological fluid. (Although the method is described with regard to a protein, other molecules (referred to herein as a target, e.g., other macromolecules, e.g., nucleic acids, can be analyzed with the methods described herein.) The method includes:

(1) providing a mixture;

(2) partitioning the mixture into to a plurality of fractions including a first and a second fraction, e.g., a soluble and a membrane fraction:

(3) contacting the target with the first fraction to form a first reaction mixture;

(4) evaluating a change in heat output of the first reaction mixture;

(5) optionally, comparing the value for heat change obtained with a predetermined value;

(6) contacting the target with the second fraction to form a second reaction mixture;

(7) evaluating a change in heat output of the second reaction mixture;

(8) optionally, comparing the value for heat change obtained with a predetermined value;

(9) evaluating, e.g., by comparing, the change in heat in the first reaction and the change in heat in the second reaction, and selecting a fraction, to thereby purify or isolate the interactor. e.g., a ligand or substrate of a target, e.g., a protein target.

In a preferred embodiment, the target, e.g., a protein, is produced by a pathogen, e.g., a prokaryotic or a eukaryotic pathogen, including a bacterium, a protozoan, a virus, e.g., phage, or a is fungus. For example, the protein can be a protein produced by any of the following species: Aquifex aeolicus, Pyrococcus horikoshii, Bacillus subtilis, Treponema pallidum, Borrelia burgdorferi, Helicobacter pylori, Archaeoglobus fulgidus, Methanobacterium thermo, Escherichia coli, Mycoplasma pneumoniae, Synechocystis sp., Methanococcus jannaschii, Saccharomyces cerevisiae, Mycoplasma genitalium, Haemophilus influenzae, Rickettsia prowazekii, Pyrococcus abyssii, Bacillus sp., Pseudomonas aeruginosa, Ureaplasma urealyticum, Pyrobaculum aerophilum, Pyrococcus furiosus, Mycobacterium tuberculosis, Mycobacterium tuberculosis, Neisseria gonorrhea, Neisseria meningiditis, Streptococcus pyogenes, Borellia burgdorferi, Caulobacter crescentus, Chlorobium tepidum, Deinococcus radiodurans, Enterococcus faecalis, Legionella pneumophila, Mycobacterium avium, Mycobacterium tuberculosis, Methanococcus jannaschii, Neisseria meningitides, Pseudomonas putida, Porphyromonas gingivalis, Salmonella typhimurium, Shewanella putrefaciens, Streptococcus pneumoniae, Vibrio cholerae, Clostridium acetobutylicum, Campylobacter jejuni, Halobacterium salinarium Institute, Listeria monocytogenes, Mycobacterium tuberculosis Sanger, Mycoplasma mycoides, Neisseria meningitidis strain, Streptomyces coelicolor, Actinobacillus actinomyce, Chlamydia trachomatis, Halobacterium sp., Mycoplasma capricolum, Neisseria gonorrhea, Pseudomonas aeruginosa, Aspergillus nidulans, Candida albicans, Leishmania major, Neurospora crassa, Pneumocystis carinii, Plasmodium falciparum, Saccharomyces cerevisiae, Schizosaccharomyces pombe, Trypanosoma cruzi, Trypanosoma brucei, Abelson murine leukemia virus, Adeno-associated virus 2 or -3, Dengue virus type 1, 2 or 3, Hepatitis A-G virus, Hepatitis GB virus B, Human T-cell lymphotropic virus type 1 or 2, Human T-cell lymphotropic virus type I, Human adenovirus type 12 or 2, Human herpesvirus 1-4, Human immunodeficiency virus type 1-2, Human parainfluenza virus 3, Human respiratory syncytial virus, Infectious hematopoietic necrosis virus, Influenza A virus, Influenza B virus, Influenza C virus and Measles virus. Additional examples of species that produce the targets tested using the methods of the invention are described below.

In a preferred embodiment, the target, e.g., a protein, is produced by a eukaryotic organism, e.g., a single-celled or a multicellular organism. Examples of such eukaryotic organisms include: Arabidopsis thaliana M, Brugia malayi, Caenorhabditis elegans, Drosophila melanogaster, Shistosoma mansoni, Shistosoma japonicum, and mammals, e.g., humans. Preferably, the target is produced by a human.

In a preferred embodiment, the target, e.g., a protein, is produced by an organelle, e.g., the mitochondria, of an organism.

In a preferred embodiment, the target, e.g., a protein, has no known activity (e.g., enzymatic activity), or has an activity which is difficult to measure.

In a preferred embodiment, the target, e.g., a protein, is a naturally-occurring protein or fragment thereof; a protein of unknown function and/or structure; a protein for which the ligand, substrate, or other interacting molecule is not known. In other embodiments, the target, e.g., a protein, has at least one enzymatic activity.

In a preferred embodiment, the target is a nucleic acid, e.g., a DNA or RNA (e.g., structured RNA, e.g., a ribozyme).

In a preferred embodiment. the method further includes repeating one or more steps, e.g., one or both of steps (4) and (5), under a different condition, e.g., at a different salt concentration, different pH, or in the presence of an exogenous cofactor.

In a preferred embodiment, the target interacts with, e.g., binds, and preferably modifies, the interactor. Modify, as used herein, includes making or breaking a bond, e.g., a non-covalent or covalent bond, in the test compound or the target. Modification includes cleavage, degradation, hydrolysis, a change in the level of phosphorylation labeling, ligation, synthesis, and similar reactions. Modification can include changes in activity, e.g., enzymatic activity, physical changes in phase, changes in aggregation, or polymerization.

In a preferred embodiment, the method further includes analyzing the interactor structure or function, e.g. analyzing the physical properties of the interactor.

In a preferred embodiment, the method further includes selecting an interactor, e.g. based on its interaction with the target, and confirming that the interactor is, e.g. a substrate or a ligand.

In a preferred embodiment, the method further includes contacting the purified or isolated interactor with a cell, e.g. a cultured cell, or an animal, and optionally determining if purified or isolated interactor has an effect on the cell or animal.

In a preferred embodiment, the method further includes selecting an interactor (e.g., a library member) on the basis of its interaction with the target and: purifying the purified or isolated interactor; crystallizing purified or isolated interactor; evaluating a physical property of the purified or isolated interactor.

In a preferred embodiment, the method further includes optimizing a property of a purified or isolated interactor, e.g., optimizing affinity for the target, altering molecular weight, e.g., decreasing molecular weight, or altering, e.g., increasing, solubility. Optimization can be performed using known methods or methods disclosed herein.

In a preferred embodiment, the change in heat output is measured with a micro calorimeter.

In a preferred embodiment, the method further includes determining a physical constant of an interaction between the target and purified or isolated interactor, e.g., k_{cat}, K_{M}. or K_{D}.

In a preferred embodiment, the method can include the use of a linking reaction, e.g., a surrogate ligand, as described elsewhere herein.

In another aspect, the invention features, a method of analyzing a target, e.g., discovering an interactor, e.g., a substrate or a ligand of a protein. The method includes:

(a) providing a reaction mixture which includes a target:

(b) contacting the target with a candidate interactor, e.g., a candidate substrate or a test ligand;

(c) evaluating a change in heat of the reaction mixture;

(d) optionally, comparing the value for heat change obtained with a predetermined value, thereby analyzing a target, e.g., discovering a substrate of the target. Although much of the discussion below is directed to proteins and their interactors, e.g., substrates or counter-ligands, it will be understood that the method can be applied to other targets and to other interactors

In a preferred embodiment, the interactor, e.g., the substrate or ligand, is identified by a change in the heat of the reaction mixture, e.g., change which is greater than a predetermined value.

In a preferred embodiment, a plurality, e.g., a library, of candidate interactors, e.g., candidate substrates or test ligands, is tested. In a preferred embodiment the plurality, e.g., a library, of candidate substrates or test ligands includes at least 10, 10², 10³, 10⁴, 10⁵, 10⁶, 10⁷, or 10⁸ candidate substrates or test ligands. Thus, in a preferred embodiment method includes:

(a) providing a reaction mixture which includes a first interactor of the plurality and the target;

(b) allowing the first interactor and the target molecule to interact;

(c) measuring a change in heat in the reaction mixture; and

(d) optionally performing steps (a), (b), and (c) for each remaining interactor of the plurality, thereby testing a plurality of interactors to determine one or more of the plurality interacts with the target.

In a preferred embodiment, the target, e.g., a protein, is produced by a pathogen, e.g., a prokaryotic or a eukaryotic pathogen, including a bacterium, a protozoan, a virus, e.g., phage, or a fungus. For example, the protein can be a protein produced by any of the following species: Aquifex acolicus, Pyrococcus horikoshii, Bacillus subtilis, Treponema pallidum, Borrelia burgdorferi, Helicobacter pylori, Archaeoglobus fulgidus, Methanobacterium thermo., Escherichia coli, Mycoplasma pneumoniae, Synechocystis sp., Methanococcus jannaschii, Saccharomyces cerevisiae, Mycoplasma genitalium, Haemophilus influenzae, Rickettsia prowazekii, Pyrococcus abyssii, Bacillus sp., Pseudomonas aeruginosa, Ureaplasma urealyticum, Pyrobaculum aerophilum, Pyrococcus furiosus, Mycobacterium tuberculosis, Mycobacterium tuberculosis, Neisseria gonorrhea, Neisseria meningiditis, Streptococcus pyogenes, Borellia burgdorferi, Caulobacter crescentus, Chlorobium tepidum, Deinococcus radiodurans, Enterococcus faecalis, Legionella pneumophila, Mycobacterium avium, Mycobacterium tuberculosis, Methanococcus jannaschii, Neisseria meningitides, Pseudomonas putida, Porphyromonas gingivalis, Salmonella typhimurium, Shewanella putrefaciens, Streptococcus pneumoniae, Vibrio cholerae, Clostridium acetobutylicum, Campylobacter jejuni, Halobacterium salinarium Institute, Listeria monocytogenes, Mycobacterium tuberculosis Sanger, Mycoplasma mycoides, Neisseria meningitidis strain, Streptomyces coelicolor, Actinobacillus actinomyce, Chlamydia trachomatis, Halobacterium sp., Mycoplasma capricolum, Neisseria gonorrhea, Pseudomonas aeruginosa, Aspergillus nidulans, Candida albicans, Leishnmania major, Neurospora crassa, Pneumocystis carinii, Plasmodium falciparum, Saccharomyces cerevisiae, Schizosaccharomyces pombe, Trypanosoma cruzi, Trypanosoma brucei, Abelson murine leukemia virus, Adeno-associated virus 2 or -3, Dengue virus type 1, 2 or 3, Hepatitis A-G virus, Hepatitis GB virus B, Human T-cell lymphotropic virus type 1 or 2, Human T-cell lymphotropic virus type I, Human adenovirus type 12 or 2, Human herpesvirus 1-4, Human immunodeficiency virus type 1-2, Human parainfluenza virus 3, Human respiratory syncytial virus, Infectious hematopoietic necrosis virus, Influenza A virus, Influenza B virus, Influenza C virus and Measles virus. Additional examples of species that produce the targets tested using the methods of the invention are described below.

In a preferred embodiment, the target, e.g., a protein, is produced by a eukaryotic organism, e.g., a single-celled or a multicellular organism. Examples of such eukaryotic organisms include: Arabidopsis thaliana M, Brugia malayi, Caenorhabditis elegans, Drosophila melanogaster, Shistosoma mansoni, Shistosoma japonicum, and mammals, e.g., humans. Preferably, the target is produced by a human.

In a preferred embodiment, the target, e.g., a protein, is produced by an organelle, e.g., the mitochondria, of an organism.

In a preferred embodiment, the target has no known activity (e.g., enzymatic activity), or has an activity which is difficult to measure. In preferred embodiments, the target has a known first activity and it is tested against a library which includes an interactor which interacts with the target by way of a second activity, e.g., an unknown activity.

In a preferred embodiment, the target is a naturally-occurring protein or fragment thereof; a protein of unknown function and/or structure; a protein for which the ligand, substrate, or other interacting molecule is not known. In other embodiments, the target, e.g., a protein, has at least one enzymatic activity.

In a preferred embodiment, the target is a nucleic acid, e.g., a DNA or RNA (e.g., structured RNA, e.g., a ribozyme).

In a preferred embodiment, a plurality of candidate interactors, e.g., library members, is tested simultaneously, e.g., in the same reaction mixture, which can allow for an increase in the throughput of the method. A plurality of library members, e.g., one which provides a positive result, can be subdivided into smaller groups and those smaller groups tested. One or more library members from the plurality or from a smaller group, e.g., one which provides a positive result, can be tested individually.

In a preferred embodiment, the method further includes repeating one or more under a different condition, e.g., at a different salt concentration, different pH, or in the presence of a different cofactor.

In a preferred embodiment, the method further includes repeating at least one step with a second or subsequent member or members of the library of candidate interactors. In a preferred embodiment, a plurality of candidate interactors, e.g., library members, is tested. In a preferred embodiment, the plurality of candidate interactors, e.g., library members, includes at least 10, 10², 10³ 10⁴ 10⁵ 10⁶ 10⁷ or 10⁸ compounds. In a preferred embodiment includes at least 10, 10², 10³, 10⁴ 10⁵, 10⁶ or 10⁸ of the library members share a structural or functional characteristic.

In a preferred embodiment, the library of candidate interactors includes a plurality of members having a common characteristic, e.g., all members of the plurality are enzyme cofactors; substrates for, e.g., biosynthetic or degradative enzymes (e.g., protease substrates), including carbohydrates, nucleoside/nucleotides, amino acids, lipids; vitamins; hormones; nucleic acids; e.g., DNA molecules; or natural products, e.g., bacterial natural products. The library can include any metabolite, precursor, or intermediate of the members listed above.

In a preferred embodiment, the library of candidate interactors is: a substrate library; a cofactor library; a carbohydrate biosynthesis and/or degradation library; a purine and pyrimidine biosynthesis and/or degradation library; an amino acid biosynthesis and/or degradation library; a lipid biosynthesis and/or degradation library; a vitamin and/or hormone library; a nucleic acid, e.g., DNA library; or a natural product library, e.g., a bacterial natural product library.

In a preferred embodiment, the candidate interactor is a species which has potential to interact with a target, e.g., a target protein. Preferably the candidate interactor is a candidate substrate or a test ligand.

In a preferred embodiment, the candidate interactor is selected from the group consisting of: an enzyme substrate, a metabolite, a cofactor, a natural product (e.g., a bacterial natural product), a carbohydrate, a polysaccharide, a nucleic acid, (e.g., a nucleoside or nucleotide precursor, a ds or ss DNA molecule, a circular nucleic acid, a super-coiled nucleic acid), an amino acid, (e.g., a D- or L-amino acid or a precursor thereof), a vitamin, a hormone, a lipid a small organic molecule, a metal, a peptide, a protein, a lipid, a glycoprotein, a glycolipid, a transition state analog and combinations thereof.

In a preferred embodiment, the method further includes testing the candidate interactor against at least one member of a second library.

In a preferred embodiment, two, or more, libraries of candidate interactors are tested simultaneously. By way of example, the target can be tested against each (or some) members of a first library, e.g., a cofactor library, and each (or some) members of a second library, e.g., a library of potential substrates. Thus, in the case of two libraries with a first library having 50 members (first₁ first₂, . . . first₅₀) and a second library having 50 members (second₁, second₂, . . . second₅₀ . . . ) the target is tested against all or a plurality of the novel combinations, e.g., against (first₁, second₁), (first₁ second₂). . . (first₁, second₅₀), and so on.

In a preferred embodiment, the member of the library of candidate interactors is a member of a combinatorial library.

In a preferred embodiment, the target interacts with, e.g., binds to, and preferably modifies, the candidate interactor. Modify, as used herein, includes making or breaking a bond, e.g., a non-covalent or covalent bond, in the candidate interactor or the target. Modification includes cleavage, degradation, hydrolysis, a change in the level of phosphorylation labeling, ligation, synthesis, and similar reactions. Modification can include changes in activity, e.g., enzymatic activity, physical changes in phase, changes in aggregation, or polymerization.

In a preferred embodiment, the method further includes analyzing the target structure or function, e.g. analyzing the physical properties of the target; analyzing the target in vitro and in vivo activity, analyzing the target (e.g. amino acid or nucleotide sequence for the presence of, e.g., conserved amino acid domains, thereby predicting the target structure or function. In a preferred embodiment, the analysis of the target structure or function is performed prior to contacting the target with the candidate interactor.

In a preferred embodiment, the method further includes selecting a candidate interactor, e.g. a library member, based on its interaction with the target; and confirming that the candidate interactor interacts with the target, e.g. is a substrate or a ligand of the target, respectively.

In a preferred embodiment, the method further includes selecting a candidate interactor, e.g. library member, based on its interaction with the target; and contacting the library member with a cell, e.g. a cultured cell, or an animal, and,. Optionally, determining if the library member has an effect on the cell or animal.

In a preferred embodiment, the method further includes selecting a candidate interactor (e.g., a library member) on the basis of its interaction with the target and: purifying the library, e.g., a candidate substrate or test ligand; crystallizing a library member, e.g., a candidate substrate or test ligand; evaluating a physical property of a library member, e.g., a candidate substrate or test ligand, e.g., molecular weight, isoelectric point, sequence (where relevant), or crystal structure.

In a preferred embodiment, the method further includes using a library member selected for interacting with the target to identify, e.g., by binding to or interacting with the selected library member, an agent which modulates an interaction between the target and the selected library member.

In a preferred embodiment, the method further includes selecting a candidate interactor (e.g., a library member) on the basis of its interaction with the target and: optimizing a property of a chosen library member, e.g., candidate substrate or test ligand, e.g., optimizing affinity for the target, altering molecular weight, e.g., decreasing molecular weight, or altering, e.g., increasing, solubility. Optimization can be performed using known methods or methods disclosed herein.

In a preferred embodiment, the change in heat output is measured with a micro calorimeter.

In a preferred embodiment, the method further includes determining a physical constant of an interaction between the protein and a member of the library, e.g., k_{cat}, K_{M}, or K_{D}.

In a preferred embodiment, the method can include the use of a linking reaction, e.g., a surrogate ligand, as described elsewhere herein.

Embodiments of the method can include the use of a linking interaction, e.g., a surrogate ligand, as is described herein. Thus, in a preferred embodiment one or more steps, e.g., step (b), further includes the inclusion of a surrogate ligand and a signal-generating entity, and the interaction of the surrogate ligand, e.g., displaced surrogate ligand, and the signal-generating entity, as described elsewhere herein.

In another aspect, the invention features, a method of modifying, e.g., optimizing, the structure of a compound. The parameter optimized can be, e.g., the ability of the compound to interact with a target, e.g., for the ability to bind or modify the target. The method includes:

(a) providing a target;

(b) modifying the structure of a test compound, e.g., by a process which involves making or breaking a bond, e.g., a covalent or non-covalent bond, to provide a modified compound

(c) contacting the target molecule with the modified compound to provide a reaction mixture;

(d) evaluating the change in heat associated with the reaction mixture;

(e) optionally, comparing the value determined in (d) with a predetermined value, thereby providing a modified compound.

In a preferred embodiment, the method includes:

(a) providing a target;

(b) contacting the target molecule with a test compound to provide a reaction mixture;

(c) determining the change in heat associated with the reaction mixture;

(d) optionally, comparing the value determined in (c) with a predetermined value, and if the value and the predetermined value manifest a predetermined relationship, e.g., if the former is equal to or less than the latter, then

(e) modifying the structure of the test compound, e.g., by making or breaking a bond, e.g., a covalent or non-covalent bond, to provide a compound, thereby providing a compound with a modified. In a preferred embodiment the method can further include one or more cycles of the following steps:

(f) contacting the target molecule with the modified test compound to provide a reaction mixture;

(g) determining the change in heat associated with the reaction mixture;

(h) optionally, comparing the value determined in (g) with a predetermined value, and if the value and the predetermined value manifest a predetermined relationship, e.g., if the former is equal to or less than the latter, then

(i) modifying the structure of the modified test compound, e.g., by making or breaking a bond, e.g., a covalent or non-covalent bond, to provide a compound.

In a preferred embodiment, the target, e.g., a protein, is produced by a pathogen, e.g., a prokaryotic or a eukaryotic pathogen, including a bacterium, a protozoan, a virus, e.g., phage, or a fungus. For example, the protein can be a protein produced by any of the following species: Aquifex aeolicus, Pyrococcus horikoshii, Bacillus subtilis, Treponema pallidum, Borrelia burgdorferi, Helicobacter pylori, Archaeoglobus fulgidus, Methanobacterium thermo, Escherichia coli, Mycoplasma pneumoniae, Synechocystis sp., Methanococcus jannaschii, Saccharomyces cerevisiae, Mycoplasma genitalium, Haemophilus influenzae, Rickettsia prowazekii, Pyrococcus abyssii, Bacillus sp., Pseudomonas aeruginosa, Ureaplasma urealyticum, Pyrobaculum aerophilum, Pyrococcus furiosus, Mycobacterium tuberculosis, Mycobacterium tuberculosis, Neisseria gonorrhea, Neisseria meningiditis, Streptococcus pyogenes, Borellia burgdorferi, Caulobacter crescentus, Chlorobium tepidum, Deinococcus radiodurans, Enterococcus faecalis, Legionella pneumophila, Mycobacterium avium, Mycobacterium tuberculosis, Methanococcus jannaschii, Neissena meningitides, Pseudomonas putida, Porphyromonas gingivalis, Salmonella typhimurium, Shewanella putrefaciens, Streptococcus pneumoniae, Vibria cholerae, Clostridium acetobutylicum, Campylobacter jejuni, Halobacterium salinarium Institute, Listeria monocytogenes, Mycobacterium tuberculosis Sanger, Mycoplasma mycoides, Neisseria meningitidis strain, Streptomyces coelicolor, Actinobacillus actinomyce, Chlamydia trachomatis, Halobacterium sp., Mycoplasma capricolum, Neisseria gonorrhea, Pseudomonas aeruginosa, Aspergillus nidulans, Candida albicans, Leishmania major, Neurospora crassa, Pneumocystis carinii, Plasmodium falciparum, Saccharomyces cerevisiae, Schizosaccharomyces pombe, Trypanosoma cruzi, Trypanosoma brucei, Abelson munne leukemia virus, Adeno-associated virus 2 or -3, Dengue virus type 1, 2 or 3, Hepatitis A-G virus, Hepatitis GB virus B, Human T-cell lymphotropic virus type 1 or 2, Human T-cell lymphotropic virus type I, Human adenovirus type 12 or 2, Human herpesvirus 1-4, Human immunodeficiency virus type 1-2, Human parainfluenza virus 3, Human respiratory syncytial virus, Infectious hematopoietic necrosis virus, Influenza A virus, Influenza B virus, Influenza C virus and Measles virus. Additional examples of species that produce the targets tested using the methods of the invention are described below.

In a preferred embodiment, the target, e.g., a protein, is produced by a eukaryotic organism, e.g., a single-celled or a multicellular organism. Examples of such eukaryotic organisms include: Arabidopsis thaliana M, Brugia malayi, Caenorhabditis elegans, Drosophila melanogaster, Shistosoma mansoni, Shistosoma japonicum, and mammals, e.g., humans. Preferably, the target is produced by a human.

In a preferred embodiment, the target, e.g., a protein, is produced by an organelle, e.g., the mitochondria, of an organism.

In a preferred embodiment, the target, e.g., protein has no known activity (e.g., enzymatic activity), or has an activity which is difficult to measure. In preferred embodiments, the target, e.g., a protein, has a known first activity and it is tested against a library which includes an interactor which interacts with the protein by way of a second activity, e.g., an unknown activity.

In-a preferred embodiment, the target is a naturally-occurring protein or fragment thereof; a protein of unknown function and/or structure; a protein for which the ligand, substrate, or other interacting molecule is not known. In other embodiments, the target, e.g., a protein, has at least one enzymatic activity.

In a preferred embodiment, the target is a nucleic acid, e.g., a DNA or RNA (e.g., structured RNA, e.g., a ribozyme).

In a preferred embodiment, the test compound (a potential or candidate interactor) is a species which has potential to interact with a target, e.g., a target protein. Preferably, the test compound is a candidate substrate or a test ligand.

In a preferred embodiment, the test compound is a member of a library that includes a plurality of members having a common characteristic, e.g., all members of the plurality are enzyme cofactors; substrates for, e.g., biosynthetic or degradative enzymes (e.g., protease substrates), including carbohydrates, nucleoside/nucleotides, amino acids, lipids; vitamins; hormones; nucleic acids; e.g., DNA molecules; or natural products, e.g., bacterial natural products. The library can include any metabolite, precursor, or intermediate of the members listed above.

In a preferred embodiment, the test compound is a member of a library selected from the group consisting of: a substrate library; a cofactor library; a carbohydrate biosynthesis and/or degradation library; a purine and pyrimidine biosynthesis and/or degradation library; an amino acid biosynthesis and/or degradation library; a lipid biosynthesis and/or degradation library; a vitamin and/or hormone library; a nucleic acid, e.g., DNA library; or a natural product library, e.g., a bacterial natural product library.

In a preferred embodiment, the test compound is selected from the group consisting of: an enzyme substrate, a metabolite, a cofactor, a natural product (e.g., a bacterial natural product), a carbohydrate, a polysaccharide, a nucleic acid, (e.g., a nucleoside or nucleotide precursor, a ds or ss DNA molecule, a circular nucleic acid, a super-coiled nucleic acid), an amino acid, (e.g., a D- or L-amino acid or a precursor thereof), a vitamin, a hormone, a lipid, a small organic molecule, a metals, a peptide, a protein, a lipid, a glycoprotein, a glycolipid, a transition state analog and combinations thereof.

In a preferred embodiment, a test compound is a member of a combinatorial library.

In a preferred embodiment, the target interacts with, e.g., binds, and preferably modifies, the test compound. Modify, as used herein, includes making or breaking a bond, e.g., a non-covalent or covalent bond, in the test compound or the target. Modification includes cleavage, degradation, hydrolysis, a change in the level of phosphorylation labeling, ligation, synthesis, and similar reactions. Modification can include changes in activity, e.g., enzymatic activity, physical changes in phase, changes in aggregation, or polymerization.

In a preferred embodiment, the method further includes analyzing the test compound, or modified test compound structure or function, e.g. analyzing the physical properties of the test compound or modified test compound; analyzing the test compound or modified test compound in vitro or in vivo activity.

In a preferred embodiment, the method further includes selecting a test compound or modified test compound, and contacting it with a cell, e.g. a cultured cell, or an animal, and, optionally, determining if the test compound or modified test compound has an effect on the cell or animal.

In a preferred embodiment, the method further includes selecting test compound or modified test compound, e.g., on the basis of its interaction with the target and: purifying the test compound or modified test compound; crystallizing test compound or modified test compound; evaluating a physical property of a test compound or modified test compound, e.g., molecular weight, isoelectric point, sequence (where relevant), or crystal structure.

In a preferred embodiment, the method further includes purifying the test compound or modified test compound, e.g., on the basis of its interaction with the target and: optimizing a property of test compound or modified test compound, e.g., optimizing affinity for the target, altering molecular weight, e.g., decreasing molecular weight, or altering, e.g., increasing, solubility. Optimization can be performed using known methods or methods disclosed herein.

In a preferred embodiment, the change in heat output is measured with a microcalorimeter.

In a preferred embodiment the method further includes determining a physical constant of an interaction between the target and the test compound or modified test compound, e.g., k_{cat}, K_{M}, or K_{D}.

In a preferred embodiment the method can include the use of a linking reaction, e.g., a surrogate ligand, as described elsewhere herein.

In another aspect, the invention features, a method of comparing two interactors, e.g., ligands, e.g., an initial ligand structure and a modification thereof. The interactors can be compared, e.g., for the ability to interact with a target, e.g., for the ability to bind or modify the target. The method includes:

(a) providing a target;

(b) contacting the target with a first interactor, e.g., a first ligand to provide a reaction mixture;

(c) determining the change in heat associated with the reaction mixture;

(d) providing a modified interactor, e.g., modified ligand, i.e., a ligand molecule in which one or more changes have been made;

(e) contacting the target with a modified interactor, e.g. modified ligand, to provide a reaction mixture;

(f) determining the change in heat associated with the reaction mixture in (e); and

(g) comparing the measurements made in (c) and (f), thereby comparing two interactors or ligands, e.g., an initial structure and a modification thereof.

In a preferred embodiment the steps can be performed in any order, e.g., (a-c) on the one hand, can be performed first, and (d-f) on the other hand, subsequently. In another preferred embodiment (a-c) on the one hand, and (d-f) on the other hand, can be performed completely or partly simultaneously.

In a preferred embodiment, the target, e.g., a protein, is produced by a pathogen, e.g., a prokaryotic or a eukaryotic pathogen, including a bacterium, a protozoan, a virus, e.g., phage, or a fungus. For example, the protein can be a protein produced by any of the following species: Aquifex aeolicus, Pyrococcus horikoshii, Bacillus subtilis, Treponema pallidum, Borrelia burgdorferi, Helicobacter pylon, Archaeoglobus fulgidus, Methanobacterium thermo., Escherichia coli, Mycoplasma pneumoniae, Synechocystis sp., Methanococcus jannaschii, Saccharomyces cerevisiae, Mycoplasma genitalium, Haemophilus influenzae, Rickettsia prowazekii, Pyrococcus abyssii, Bacillus sp., Pseudomonas aeruginosa, Ureaplasma urealyticum, Pyrobaculum aerophilum, Pyrococcus furiosus, Mycobacterium tuberculosis, Mycobacterium tuberculosis, Neisseria gonorrhea, Neisseria meningiditis, Streptococcus pyogenes, Borellia burgdorferi, Caulobacter crescentus, Chlorobium tepidum, Deinococcus radiodurans, Enterococcus faecalis, Legionella pneumophila, Mycobacterium avium, Mycobacterium tuberculosis, Methanococcus jannaschii, Neisseria meningitides, Pseudomonas putida, Porphyromonas gingivalis, Salmonella typhimurium, Shewanella putrefaciens, Streptococcus pneumoniae, Vibrio cholerae, Clostridium acetobutylicum, Campylobacter jejuni, Halobacterium salinarium Institute, Listeria monocytogenes, Mycobacterium tuberculosis Sanger, Mycoplasma mycoides, Neisseria meningitidis strain, Streptomyces coelicolor, Actinobacillus actinomyce, Chlamydia trachomatis, Halobacterium sp., Mycoplasma capricolum, Neisseria gonorrhea, Pseudomonas aeruginosa, Aspergillus nidulans, Candida albicans, Leishmania major, Neurospora crassa, Pneumocystis carinii, Plasmodium falciparum, Saccharomyces cerevisiae, Schizosaccharomyces pombe, Trypanosoma cruzi, Trypanosoma brucei, Abelson murine leukemia virus, Adeno-associated virus 2 or -3, Dengue virus type 1, 2 or 3, Hepatitis A-G virus, Hepatitis GB virus B, Human T-cell lymphotropic virus type 1 or 2, Human T-cell lymphotropic virus type I, Human adenovirus type 12 or 2, Human herpesvirus 1-4, Human immunodeficiency virus type 1-2, Human parainfluenza virus 3, Human respiratory syncytial virus, Infectious hematopoietic necrosis virus, Influenza A virus, Influenza B virus, Influenza C virus and Measles virus. Additional examples of species that produce the targets tested using the methods of the invention are described below.

In a preferred embodiment, the target, e.g., a protein, is produced by a eukaryotic organism, e.g., a single-celled or a multicellular organism. Examples of such eukaryotic organisms include: Arabidopsis thaliana M, Brugia malayi, Caenorhabditis elegans, Drosophila melanogaster, Shistosoma mansoni, Shistosoma japonicum, and mammals, e.g., humans. Preferably, the target is produced by a human.

In a preferred embodiment, the target, e.g., a protein, is produced by an organelle, e.g., the mitochondria, of an organism.

In a preferred embodiment, the target, e.g., a protein, has no known activity (e.g., enzymatic activity), or has an activity which is difficult to measure.

In a preferred embodiment, the target is a naturally occurring protein or fragment thereof; a protein of unknown function and/or structure; a protein for which the ligand, substrate, or other interacting molecule is not known. In other embodiments, the target e.g., a protein, has at least one enzymatic activity.

In a preferred embodiment, the target is a nucleic acid, e.g., a DNA or RNA (e.g., structured RNA, e.g., a ribozyme).

In a preferred embodiment, the method further includes repeating one or more steps under a different condition, e.g., at a different salt concentration, different pH, or in the presence of a different cofactor.

In a preferred embodiment, the target interacts with, e.g., binds, and preferably modifies, the interactor. Modify, as used herein, includes making or breaking a bond, e.g., a non-covalent or covalent bond, in the test compound or the target. Modification includes cleavage, degradation, hydrolysis, a change in the level of phosphorylation labeling, ligation, synthesis, and similar reactions. Modification can include changes in activity, e.g., enzymatic activity, physical changes in phase, changes in aggregation, or polymerization.

In a preferred embodiment, the method further includes analyzing an interactor structure or function, e.g. analyzing the physical properties of an interactor; analyzing an interactor in vitro or in vivo activity.

In a preferred embodiment, the method further includes selecting an interactor, e.g. based on its interaction with the target; and contacting an interactor with a cell, e.g. a cultured cell, or an animal, and, optionally determining if interactor has an effect on the cell or animal.

In a preferred embodiment, the method further includes selecting an interactor, e.g., on the basis of its interaction with the target and an interactor; an interactor; evaluating a physical property of an interactor, e.g., molecular weight, isoelectric point, sequence (where relevant), or crystal structure.

In a preferred embodiment, the method further includes selecting an interactor on the basis of its interaction with the target and: optimizing a property of the interactor, e.g., optimizing affinity for the target, altering molecular weight, e.g., decreasing molecular weight, or altering, e.g., increasing, solubility. Optimization can be performed using known methods or methods disclosed herein.

In a preferred embodiment, the change in heat output is measured with a microcalorimeter.

In a preferred embodiment, the method further includes determining a physical constant of an interaction between the protein and an interactor, e.g., k_{cat}, K_{M}, or K_{D}.

In a preferred embodiment, the method can include the use of a linking reaction, e.g., a surrogate ligand, as described elsewhere herein.

In another aspect, the invention features, a method of comparing a subject molecule and a modification thereof, e.g., an initial structure and a modification thereof. The method includes:

(a) providing a subject molecule;

(b) allowing the subject molecule to undergo an interaction with a second molecule, e.g., binding, with a ligand, or an interaction between a first moiety of the subject molecule and a second moiety of the subject molecule;

(c) determining the change in heat associated with the interaction;

(d) providing a modified subject molecule in which one or more changes have been made;

(e) allowing the modified subject molecule to undergo an interaction with a second molecule, e.g., binding, with a ligand, or an interaction between a first moiety of the subject molecule and a second moiety of the subject molecule;

(f) determining the change in heat associated with the interaction in (e); and

(g) comparing (c) and (f), thereby comparing a subject molecule and a modification thereof

In a preferred embodiment the steps can be performed in any order, e.g., (a-c) on the one hand, can be performed first and (d-f) on the other hand, subsequently or, (a-c) on the one hand, and (d-f) on the other hand, can be performed completely or partly simultaneously.

In a preferred embodiment, the method further includes selecting a modified molecule which interacts with the target; and confirming that the modified molecule interacts with, e.g. binds, to the target in a second test, e.g. one in which the surrogate ligand is not present.

In a preferred embodiment, the method further includes selecting a modified molecule that interacts with the target; and confirming that the modified molecule interacts with, e.g. binds to the target by contacting the modified molecule with the target in vitro, e.g. in the absence of the surrogate modified molecule.

In preferred embodiments, the method further includes selecting a modified molecule which interacts with the target; and contacting the ligand with a cell, e.g., a cultured cell, or an animal, and, optionally, determining if the ligand has an effect on the cell or animal.

In a preferred embodiment the method further includes: purifying a test ligand; crystallizing a test ligand; evaluating a physical property of a test ligand, e.g., molecular weight, isoelectric point, sequence (where relevant), or crystal structure.

In a preferred embodiment the method further includes: optimizing a property of a chosen test ligand, e.g., optimizing affinity for the target, altering molecular weight, e.g., decreasing molecular weight, or altering, e.g., increasing, solubility. Optimization can be performed using known methods or methods disclosed herein, In a preferred embodiment the change in heat is measured with a microcalorimeter.

In another aspect, the invention features, a method of analyzing a compound, e.g., a protein or nucleic acid, e.g., a structured RNA, or other target. The method includes:

(a) providing reaction mixture which includes a target; (b) inducing a conformational change in the target in the absence of an interaction or entity, e.g., the absence of an interaction with a ligand (or other library member),or in the absence of a ligand (or other library member);

(c) measuring the change in heat evolved in said conformational change;

(d) inducing a conformational change in the target in the presence of an interaction or entity, e.g., the presence of an interaction with a ligand (or other library member) or in the presence of a ligand (or other library member);

(e) measuring the change in heat evolved in said conformational change; and

(g) comparing the value obtained in (c) with the value obtained in (e) thereby analyzing a target.

In a preferred embodiment a denaturant, e.g., guanidine hydrochloride, urea, or a similar agent, is added to the reaction mixture.

In a preferred embodiment the measurement is made with a microcalorimeter.

In a preferred embodiment the method includes:

(a) providing reaction mixture which includes a target;

(b) adding heat to the reaction mixture in the absence of an interaction or entity, e.g., the absence of an interaction with a ligand or in the absence of a ligand;

(c) measuring the change in heat evolved in said conformational change, e.g., to obtain an apparent specific heat output;

(d) adding heat to the reaction mixture in the presence of an interaction or entity, e.g., the presence of an interaction with a ligand or in the presence of a ligand;

(e) measuring the change in heat evolved in said conformational change, e.g., to obtain an apparent specific heat output; and

(g) comparing the value obtained in (c) with the value obtained in (e), thereby analyzing a target, e.g., to determine the binding of a ligand to the target.

In a preferred embodiment, the target is a protein or polypeptide, a nucleic acid, e.g., an RNA. It can be purified, partially purified, or in a crude state.

In a preferred embodiment, a denaturant, e.g., guanidine hydrochloride, urea, or a similar agent, is added to the reaction mixture.

In a preferred embodiment, one or more conditions, e.g., the concentration of the denaturant and the target, or the temperature, is chosen such the presence of a ligand that binds the relatively more compactly folded state results in a relatively large change in heat, e.g., by driving the target molecules into the folded state.

In a preferred embodiment, the change in heat is measured with a microcalorimeter.

In a preferred embodiment, the target, e.g., a protein, is produced by a pathogen, e.g., a prokaryotic or a eukaryotic pathogen, including a bacterium, a protozoan, a virus, e.g., phage, or a fungus. For example, the protein can be a protein produced by any of the following species: Aquifex aeolicus, Pyrococcus horikoshii, Bacillus subtilis, Treponema pallidum, Borrelia burgdorferi, Helicobacter pylori, Archaeoglobus fulgidus, Methanobacterium thermo., Escherichia coli, Mycoplasma pneumoniae, Synechocystis sp., Methanococcus jannaschii, Saccharomyces cerevisiae, Mycoplasma genitalium, Haemophilus influenzae, Rickettsia prowazekii, Pyrococcus abyssii, Bacillus sp., Pseudomonas aeruginosa, Ureaplasma urealyticum, Pyrobaculum aerophilum, Pyrococcus funosus, Mycobacterium tuberculosis, Mycobacterium tuberculosis, Neisseria gonorrhea, Neisseria meningiditis, Streptococcus pyogenes, Borellia burgdorferi, Caulobacter crescentus, Chlorobium tepidum, Deinococcus radiodurans, Enterococcus faecalis, Legionella pneumophila, Mycobacterium avium, Mycobacterium tuberculosis, Methanococcus jannaschii, Neisseria meningitides, Pseudomonas putida, Porphyromonas gingivalis, Salmonella typhimurium, Shewanella putrefaciens, Streptococcus pneumoniae, Vibrio cholerae, Clostridium acetobutylicum, Campylobacter jejuni, Halobacterium salinarium Institute, Listeria monocytogenes, Mycobacterium tuberculosis Sanger, Mycoplasma mycoides, Neisseria meningitidis strain, Streptomyces coelicolor, Actinobacillus actinomyce, Chlamydia trachomatis, Halobacterium sp., Mycoplasma capricolum, Neisseria gonorrhea, Pseudomonas aeruginosa, Aspergillus nidulans, Candida albicans, Leishmania major, Neurospora crassa, Pneumocystis carinii, Plasmodium falciparum, Saccharomyces cerevisiae, Schizosaccharomyces pombe, Trypanosoma cruzi, Trypanosoma brucei, Abelson murine leukemia virus, Adeno-associated virus 2 or -3, Dengue virus type 1, 2 or 3, Hepatitis A-G virus, Hepatitis GB virus B, Human T-cell lymphotropic virus type 1 or 2, Human T-cell lymphotropic virus type I, Human adenovirus type 12 or 2, Human herpesvirus 1-4, Human immunodeficiency virus type 1-2, Human parainfluenza virus 3, Human respiratory syncytial virus, Infectious hematopoietic necrosis virus, Influenza A virus, Influenza B virus, Influenza C virus and Measles virus. Additional examples of species that produce the targets tested using the methods of the invention are described below.

In a preferred embodiment, the target, e.g., a protein, is produced by a eukaryotic organism, e.g., a single-celled or a multicellular organism. Examples of such eukaryotic organisms include: Arabidopsis thaliana M, Brugia malayi, Caenorhabditis elegans, Drosophila melanogaster, Shistosoma mansoni, Shistosoma japonicum, and mammals, e.g., humans. Preferably, the target is produced by a human.

In a preferred embodiment, the target, e.g., a protein, is produced by an organelle, e.g., the mitochondria, of an organism.

In a preferred embodiment, the target, e.g., the protein, has no known activity (e.g., enzymatic activity), or has an activity which is difficult to measure. In preferred embodiments, the target, e.g., protein, has a known first activity and it is tested against a library which includes an interactor which interacts with the protein by way of a second activity, e.g., an unknown activity.

In a preferred embodiment, the target is a naturally occurring protein or fragment thereof, a protein of unknown function and/or structure; a protein for which the ligand, substrate, or other interacting molecule is not known. In other embodiments, the target, e.g., the protein, has at least one enzymatic activity.

In a preferred embodiment, the target is a nucleic acid, e.g., a DNA or RNA (e.g., structured RNA, e.g., a ribozyme).

In a preferred embodiment, a plurality of library members is tested simultaneously, e.g., in the same reaction mixture, which can allow for an increase in the throughput of the method. A plurality of library members, e.g., one which provides a positive result, can be subdivided into smaller groups and those smaller groups tested. One or more library members from the plurality or from a smaller group, e.g., one which provides a positive result, can be tested individually.

In a preferred embodiment, the method further includes repeating one or more steps under a different condition, e.g., at a different salt concentration, different pH, or in the presence of a different cofactor.

In a preferred embodiment, the method further includes repeating at least one with a second or subsequent member or members of the library. In a preferred embodiment, a plurality of library members, e.g., candidate substrates or test ligands, is tested. In a preferred embodiment, the plurality of library members includes at least 10, 10², 10³, 10⁴, 10⁵, 10⁶, 10⁷, or 10⁸ compounds. In a preferred embodiment includes at least 10, 10², 10³, 10⁴, 10⁵, 10⁶, 10⁷, or 10⁸ of the library members share a structural or functional characteristic.

In a preferred embodiment, the library includes a plurality of members having a common characteristic, e.g., all members of the plurality are enzyme cofactors; substrates for, e.g., biosynthetic or degradative enzymes (e.g., protease substrates), including carbohydrates, nucleoside/nucleotides, amino acids, lipids; vitamins; hormones; nucleic acids; e.g., DNA molecules; or natural products, e.g., bacterial natural products. The library can include any metabolite, precursor, or intermediate of the members listed above.

In a preferred embodiment, the library is: a substrate library; a cofactor library; a carbohydrate biosynthesis and/or degradation library; a purine and pyrimidine biosynthesis and/or degradation library; an amino acid biosynthesis and/or degradation library; a lipid biosynthesis and/or degradation library; a vitamin and/or hormone library; a nucleic acid, e.g., DNA library; or a natural product library, e.g., a bacterial natural product library.

In a preferred embodiment, a library member (a potential or candidate interactor) is a species which has potential to interact with a target, e.g., a target protein. Preferably, a library member is a candidate substrate or a test ligand.

In a preferred embodiment, a library member is selected from the group consisting of: an enzyme substrate, a metabolite, a cofactor, a natural product (e.g., a bacterial natural product), a carbohydrate, a polysaccharide, a nucleic acid, (e.g., a nucleoside or nucleotide precursor, a ds or ss DNA molecule, a circular nucleic acid, a super-coiled nucleic acid), an amino acid, (e.g., a D- or L-amino acid or a precursor thereof), a vitamin, a hormone, a lipid, a small organic molecule, a metals, a peptide, a protein, a lipid, a glycoprotein, a glycolipid, a transition state analog and combinations thereof.

In a preferred embodiment, the method further includes testing the protein against at least one member of a second library.

In a preferred embodiment, two, or more, libraries are tested simultaneously. By way of example, the target can be tested against each (or some) members of a first library, e.g., a cofactor library, and each (or some) members of a second library, e.g., a library of potential substrates. Thus, in the case of two libraries with a first library having 50 members (first₁, first₂, . . . first₅₀) and a second library having 50 members (second₁, second₂, . . . second₅₀ . . . ) the target is tested against all or a plurality of the novel combinations, e.g., against (first₁, second₁), (first₁ second₂) . . . (first₁, second₅₀), and so on.

In a preferred embodiment, a library member is a member of a combinatorial library.

In a preferred embodiment, the target interacts with, e.g., binds, and preferably modifies, the library member. Modify, as used herein, includes making or breaking a bond, e.g., a non-covalent or covalent bond, in the test compound or the target. Modification includes cleavage, degradation, hydrolysis, a change in the level of phosphorylation labeling, ligation, synthesis, and similar reactions. Modification can include changes in activity, e.g., enzymatic activity, physical changes in phase, changes in aggregation, or polymerization.

In a preferred embodiment, the method further includes:

analyzing library member structure or function, e.g., analyzing the physical properties of the target; analyzing library member in vitro or in vivo activity.

In a preferred embodiment, the method further includes:

selecting a library member, e.g., candidate substrate or test ligand based on its interaction with the target; and confirming that the candidate substrate or test ligand is a substrate or a ligand, respectively.

In a preferred embodiment, the method further includes:

selecting a library member based on its interaction with the target; and contacting the library member with a cell, e.g., a cultured cell, or an animal, and, optionally, determining if the library member has an effect on the cell or animal.

In a preferred embodiment, the method further includes selecting an interactor (e.g., a library member) on the basis of its interaction with the target and: purifying the library, e.g., a candidate substrate or test ligand; crystallizing a library member, e.g., a candidate substrate or test ligand; evaluating a physical property of a library member, e.g., a candidate substrate or test ligand, e.g., molecular weight, isoelectric point, sequence (where relevant), or crystal structure.

In a preferred embodiment, the method further includes using a library member selected for interacting with the target to identify, e.g., by binding to or interacting with the selected library member, an agent which modulates an interaction between the target and the selected library member.

In a preferred embodiment, the method further includes selecting an interactor (e.g., a library member) on the basis of its interaction with the target and: optimizing a property of a chosen library member, e.g., candidate substrate or test ligand, e.g., optimizing affinity for the target, altering molecular weight, e.g., decreasing molecular weight, or altering, e.g., increasing, solubility. Optimization can be performed using known methods or methods disclosed herein.

In a preferred embodiment, the change in heat output is measured with a microcalorimeter.

In a preferred embodiment, the method further includes determining a physical constant of an interaction between the protein and a member of the library, e.g., k_{cat}, K_{M}, or k_{D}.

In another aspect, the invention features, a method of analyzing an interactor, e.g., a substrate, e.g., discovering a target molecule which modifies the substrate. The method includes: providing a reaction mixture which includes the interactor, e.g., substrate:

contacting the interactor with a candidate target;

evaluating a change in heat the reaction mixture;

optionally, comparing the value for heat change obtained with a predetermined value, thereby of analyzing a interactor, e.g., discovering a target for the interactor.

In a preferred embodiment, the interactor is identified by a change in the heat of the reaction mixture, e.g., change which is greater than a predetermined value.

In a preferred embodiment, a plurality of candidate targets are tested. In a preferred embodiment the plurality of candidate targets includes at least 10, 10², 10³ 10⁴ 10⁵ 10⁶ 10⁷, or 10⁸ candidate targets.

In a preferred embodiment, the target interacts with, e.g., binds, and preferably modifies, the substrate. Modify includes making or breaking a bond, e.g., a non-covalent or covalent bond, in the surrogate ligand (or in the signal-generating entity itself). Modification includes cleavage, degradation, hydrolysis, a change in the level of phosphorylation labeling, ligation, synthesis, and similar reactions. Modification can include physical changes in phase, changes in aggregation, or polymerization.

In a preferred embodiment, the method further includes:

selecting a candidate target; and confirming that candidate target modified the target.

In a preferred embodiment, the method further includes:

selecting a candidate target; and contacting the candidate target with a cell, e.g., a cultured cell, or an animal, and, optionally, determining if the candidate target has an effect on the cell or animal.

In a preferred embodiment, the method further includes: purifying a candidate target; crystallizing a candidate target; evaluating a physical property of a candidate target, e.g., molecular weight, isoelectric point, sequence (where relevant), or crystal structure.

In a preferred embodiment, the method further includes: optimizing a property of a chosen candidate target, e.g., optimizing affinity for the substrate, altering molecular weight, e.g., decreasing molecular weight, or altering, e.g., increasing, solubility. Optimization can be performed using known methods or methods disclosed herein,

In a preferred embodiment, the change in heat is measured with a microcalorimeter.

In another aspect, the invention features, a method of analyzing a target, e.g., analyzing an interaction of a target and a second entity. The method includes:

a change in conformation;

allowing a product of the linking reaction to enter a second reaction, e.g., the cleavage or degradation of a surrogate ligand; and measuring the heat change from the second reaction, thereby analyzing an interaction of the target.

In a preferred embodiment the linking reaction or the second reaction can include a change in phase.

In a preferred embodiment one or more additional reactions can be interposed between the linking reaction and the second reaction.

In a preferred embodiment the interaction of the target can be, e.g., an interaction between the target and another molecule, e.g., a ligand or a solute molecule, or an interaction of a first moiety of the target with a second moiety of the target, e.g., autophosphorylation, or a change in the conformation of the target, e.g., the secondary, tertiary, or quaternary, structure of the target.

In a preferred embodiment: the reaction mixture is not transparent; the reaction mixture is colored; the reaction mixture is turbid; the reaction mixture contains a substance which interferes with fluorescent or colorimetric detection; the reaction mixture is not a pure solution, e.g., it contains products other than the target. In a preferred embodiment the reaction mixture contains: a substance which interferes with radioactive analysis; a substance which interferes with spectrophotometric analysis, e.g., NMR analysis.

In a preferred embodiment the change in heat is measured with a microcalorimeter.

In another aspect, the invention features, a method of analyzing a test ligand, target, or an interaction between the two. The method includes: providing a reaction

mixture containing a surrogate ligand and a target, contacting the reaction

mixture with the test ligand and with a signal-generating entity; wherein

the signal-generating entity is present with the surrogate ligand under conditions which allow it to interact with surrogate ligand, e.g., with surrogate ligand which has been displaced from the target by binding of the test ligand to the target; and measuring

the change in heat in the reaction mixture, thereby analyzing a test

ligand, target, or an interaction between the two.

In preferred embodiments, the surrogate ligand exhibits negative heterotropic linkage with respect to a test ligand which can bind the target, (i.e., it is displaced upon binding of the test ligand to the target). This is the preferred embodiment and the subject of most of the discussion herein. However, the invention also includes embodiments wherein the surrogate ligand binds the target upon binding of the test ligand, thereby reducing the level of free surrogate ligand and thereby providing less surrogate ligand to interact with the signal-generating entity.

In preferred embodiments, the interaction between the signal-generating entity and the surrogate ligand occurs more readily between the signal-generating entity and free (as opposed to target-bound) surrogate ligand. By way of example the interaction between the signal-generating entity and free (as opposed to target-bound) surrogate ligand occurs at least 2, 5, 10, 10², 10³ 10⁴ 10⁵, 10⁶, 10⁷ or 10⁸ fold more readily between the signal-generating entity and free (as opposed to target-bound) surrogate ligand. However, the invention also includes embodiments wherein the interaction between the signal-generating entity and the surrogate ligand occurs more readily between the signal-generating entity and target-bound (as opposed to free) surrogate ligand. By way of example the interaction between the signal-generating entity and target-bound (as opposed to free) surrogate ligand occurs at least 2, 5, 10, 10², 10³, 10⁴, 10⁵, 10⁶, 10⁷, or 10⁸ fold more readily between the signal-generating entity and target-bound (as opposed to free) surrogate ligand.

In a preferred embodiment, the surrogate ligand is an ion, e.g., a proton. In a preferred embodiment the signal-generating entity is a buffer molecule, e.g., with a relatively large heat of ionization, e.g., Tris-HCl.

In a preferred embodiment, the surrogate ligand is a factor which modulates, e.g., increases or decreases, the activity of the signal-generating entity. By way of example, the surrogate ligand can be a metal ion which activates (or inhibits) an enzyme which is the signal-generating entity.

In a preferred embodiment, the signal-generating entity interacts with, e.g., binds, and preferably modifies, surrogate ligand, e.g., free surrogate ligand. Modify includes making or breaking a bond, e.g., a non-covalent or covalent bond, in the surrogate ligand (or in the signal-generating entity itself). Modification includes cleavage, degradation, hydrolysis, a change in the level of phosphorylation labeling, ligation, synthesis, and similar reactions. Modification can also include physical changes in phase, changes in aggregation, or polymerization.

In a preferred embodiment, the target is a protein or polypeptide, the surrogate ligand is a nucleic acid, the signal-generating entity is an enzyme which cleaves a bond in a nucleic acid, e.g., a nuclease.

In a preferred embodiment, the method further includes:

In a preferred embodiment, the method further includes:

selecting a test ligand which interacts with the target; and confirming that the test ligand interacts with, e.g., binds, to the target by contacting the ligand with the target in vitro, e.g., in the absence of the surrogate ligand.

In a preferred embodiment, the method further includes:

selecting a ligand which interacts with the target; and contacting the ligand with a cell, e.g., a cultured cell, or an animal, and, optionally, determining if the ligand has an effect on the cell or animal.

In a preferred embodiment, the method further includes:

purifying a test ligand; crystallizing a test ligand; evaluating a physical property of a test ligand, e.g., molecular weight, isoelectric point, sequence (where relevant), or crystal structure.

In a preferred embodiment the method further includes:

optimizing a property of a chosen test ligand, e.g., optimizing affinity for the target, altering molecular weight , e.g., decreasing molecular weight, or altering, e.g., increasing, solubility. Optimization can be performed using known methods or methods disclosed herein,

In a preferred embodiment, the surrogate ligand, e.g., a surrogate ligand, e.g., a nucleic acid, is amplified, e.g., with PCR or more preferably with an isothermal amplification method, prior to interaction with the signal-generating entity.

In a preferred embodiment the change in heat is measured with a microcalorimeter.

In a preferred embodiment, the signal-generating entity interacts directly with the surrogate ligand. In other embodiments it interacts indirectly, e.g., it interacts with an amplification product generated from the surrogate ligand or it acts on the product of a reaction between the surrogate ligand and another entity.

In another aspect, the invention features, a method of analyzing a target, a test ligand, or the interaction between the two. The method includes:

providing a reaction mixture containing a surrogate ligand and the target;

contacting the reaction mixture with the test ligand and with a signal-generating entity;

wherein the signal-generating entity is present with the surrogate ligand under conditions which allow it to interact with free surrogate ligand, e.g., with surrogate ligand which has been displaced from the target by binding of the test ligand; and measuring the change in heat in the reaction mixture, thereby analyzing a test ligand, target, or an interaction between the two.

In preferred embodiments, the surrogate ligand exhibits negative heterotropic linkage with respect to a test ligand which can bind the target, (i.e., it is displaced upon binding of the test ligand to the target). This is the preferred embodiment and the subject of most of the discussion herein. However, the invention also includes embodiments wherein the surrogate ligand binds the target upon binding of the test ligand, thereby reducing the level of free surrogate ligand and thereby providing less surrogate ligand to interact with the signal-generating entity.

In preferred embodiments, the interaction between the signal-generating entity and the surrogate ligand occurs more readily between the signal-generating entity and free (as opposed to target-bound) surrogate ligand. By way of example the interaction between the signal-generating entity and free (as opposed to target-bound) surrogate ligand occurs at least 2, 5, 10, 10², 10³, 10⁴, 10⁵, 10⁶, 10⁷, or 10⁸ fold more readily between the signal-generating entity and free (as opposed to target-bound) surrogate ligand. However, the invention also includes embodiments wherein the interaction between the signal-generating entity and the surrogate ligand occurs more readily between the signal-generating entity and target-bound (as opposed to free) surrogate ligand. By way of example the interaction between the signal-generating entity and target-bound (as opposed to free surrogate ligand occurs at least 2, 5, 10, 10², 10³, 10⁴, 10⁵, 10⁶, 10⁷, or 10⁸ fold more readily between the signal-generating entity and target-bound (as opposed to free) surrogate ligand.

In a preferred embodiment, the signal-generating entity interacts with, e.g., binds, and preferably modifies, free surrogate ligand. Modify includes making or breaking a bond, e.g., a non-covalent or covalent bond, in the surrogate ligand (or in the signal-generating entity itself). Modification includes cleavage, degradation, hydrolysis, a change in the level of phosphorylation labeling, ligation, synthesis, and similar reactions. Modification can include physical changes in phase, changes in aggregation, or polymerization.

In a preferred embodiment, the signal-generating entity is a degradative enzyme.

In a preferred embodiment, the surrogate ligand is a nucleic acid and the signal-generating entity is an enzyme which modifies a nucleic acid, or uses the nucleic acid for a substrate or template, e.g., the signal-generating entity an enzyme, e.g., a nuclease, e.g., a DNAse, e.g., an endonuclease or an exonuclease, a polymerase, e.g., a DNA polymerase: the signal-generating entity modifies a protein, e.g., by making or breaking a covalent or non-covalent bond in the surrogate ligand (or itself) e.g., it cleaves a peptide bond, e.g., is a protease, and the surrogate ligand includes a peptide bond, e.g., is a protein.

In a preferred embodiment, the method further includes:

selecting a ligand which interacts with the target; and confirming that the test ligand interacts with, e.g., binds, to the target in a second test, e.g., one in which the surrogate ligand is not present.

In a preferred embodiment, the method further includes:

selecting a test ligand which interacts with the target; and confirming that the ligand interacts with, e.g., binds, to the target by contacting the test ligand with the target in vitro, e.g., in the absence of the surrogate ligand.

In a preferred embodiment, the method further includes:

selecting a test ligand which interacts with the target; and contacting the test ligand with a cell, e.g., a cultured cell, or an animal, and, optionally, determining if the test ligand has an effect on the cell or animal.

In a preferred embodiment, the method further includes: purifying a test ligand; crystallizing a test ligand; evaluating a physical property of a test ligand, e.g., molecular weight, isoelectric point, sequence (where relevant), or crystal structure.

In a preferred embodiment, the method further includes: optimizing a property of a chosen test ligand, e.g., optimizing affinity for the target, altering molecular weight, e.g., decreasing molecular weight, or altering, e.g., increasing, solubility. Optimization can be performed using known methods or methods disclosed herein.

In a preferred embodiment, the reaction mixture is not transparent; the reaction mixture is colored; the reaction mixture is turbid; the reaction mixture contains a substance which interferes with fluorescent or colorimetric detection; the reaction mixture is not a pure solution, e.g., it contains products other than the target. In a preferred embodiment the reaction mixture contains: a substance which interferes with radioactive analysis; a substance which interferes with spectrophotometric analysis, e.g., NMR analysis.

In a preferred embodiment, the surrogate ligand, e.g., a nucleic acid, is amplified, e.g., with PCR or more preferably with an isothermal amplification method, prior to interaction with the signal-generating entity.

In a preferred embodiment, the change in heat is measured with a micro calorimeter.

In a preferred embodiment, the signal-generating entity interacts directly with the surrogate ligand. In other embodiments is interacts indirectly, e.g., it interacts with an amplification product generated from the surrogate ligand or it acts on the product of a reaction between the surrogate ligand and another entity.

In another aspect, the invention features, a method of analyzing a target, a test ligand, or the interaction between the two. The method includes:

providing a reaction mixture containing a surrogate ligand, which is a nucleic acid, and the target; contacting the reaction mixture with the test ligand and with a signal-generating entity, which is a molecule which makes or breaks a bond, e.g., a covalent or non-covalent bond, in the surrogate ligand;

wherein the signal-generating entity is present with the surrogate ligand under conditions which allow it to interact with free surrogate ligand, e.g., with surrogate ligand which has been displaced from the target by binding of the test ligand; and

measuring the change in heat in the reaction mixture, thereby analyzing a test ligand, target, or an interaction between the two.

In preferred embodiments, the surrogate ligand exhibits negative heterotropic linkage with respect to a test ligand which can bind the target, (i.e., it is displaced upon binding of the test ligand to the target).

In preferred embodiments, the interaction between the signal-generating entity and the surrogate ligand occurs more readily between the signal-generating entity and free (as opposed to target-bound) surrogate ligand. By way of example the interaction between the signal-generating entity and free (as opposed to target-bound) surrogate ligand occurs at least 2, 5, 10, 10², 10³, 10⁴, 10⁵, 10⁶, 10⁷, or 10⁸ fold more readily between the signal-generating entity and free (as opposed to target-bound) surrogate ligand.

In a preferred embodiment, the signal-generating entity is a degradative enzyme.

In a preferred embodiment, the signal-generating entity is an enzyme which modifies a nucleic acid, or uses the nucleic acid for a substrate or template, e.g., the signal-generating entity an enzyme, e.g., a nuclease, e.g., an endonuclease or an exonuclease, a polymerase, e.g., a DNA polymerase.

In a preferred embodiment, the method further includes:

selecting a test ligand which interacts with the target; and confirming that the test ligand interacts with, e.g., binds, to the target in a second test, e.g., one in which the surrogate ligand is not present.

In a preferred embodiment, the method further includes:

selecting a ligand which interacts with the target; and confirming that the test ligand interacts with, e.g., binds, to the target by contacting the test ligand with the target in vitro, e.g., in the absence of the surrogate ligand.

In a preferred embodiment, the method further includes:

selecting a test ligand which interacts with the target; and contacting the test ligand with a cell, e.g., a cultured cell, or an animal, and, optionally, determining if the ligand has an effect on the cell or animal.

In a preferred embodiment, the method further includes: purifying a test ligand; crystallizing a test ligand; evaluating a physical property of a test ligand, e.g., molecular weight, isoelectric point, sequence (where relevant), or crystal structure.

In a preferred embodiment, the method further includes: optimizing a property of a chosen test ligand, e.g., optimizing affinity for the target, altering molecular weight, e.g., decreasing molecular weight, or altering, e.g., increasing, solubility. Optimization can be performed using known methods or methods disclosed herein.

In a preferred embodiment, the surrogate ligand is amplified, e.g., with PCR or more is preferably with an isothermal amplification method, e.g., prior to interaction with the signal-generating entity.

In a preferred embodiment, the change in heat is measured with a microcalorimeter.

In a preferred embodiment, the signal-generating entity interacts directly with the surrogate ligand. In other embodiments it interacts indirectly, e.g., it interacts with an amplification product generated from the surrogate ligand or it acts on the product of a reaction between the surrogate ligand and another entity.

In another aspect, the invention features a library of interaction candidates, e.g., a library of candidate substrates or test ligands as described herein. Preferably, the library includes at least one member which is known to interact with a target.

In a preferred embodiment, the library is: a substrate library; a cofactor library; a carbohydrate biosynthesis and/or degradation library; a purine and pyrimidine biosynthesis and/or degradation library; an amino acid biosynthesis and/or degradation library; a lipid biosynthesis and/or degradation library; a vitamin and/or hormone library; a nucleic acid, e.g., DNA library; or a natural product library, e.g., a bacterial natural product library.

In a preferred embodiment, a library member is a species which has potential to interact with a target, e.g., a target protein. Preferably, a library member is a candidate substrate or a test ligand.

In a preferred embodiment, a library member is selected from the group consisting of: an enzyme substrate, a metabolite, a cofactor, a natural product (e.g., a bacterial natural product), a carbohydrate, a polysaccharide, a nucleic acid (e.g., a nucleoside or nucleotide precursor, a double-stranded (ds) or single-stranded (ss) DNA molecule, a circular nucleic acid, a super-coiled nucleic acid), an amino acid, (e.g., a D- or L-amino acid or a precursor thereof), a vitamin, a hormone, a lipid, a small organic molecule, a metals, a peptide, a protein, a lipid, a glycoprotein, a glycolipid, a transition state analog and combinations thereof.

In a preferred embodiment, the library includes a plurality of members having a common characteristic, e.g., all members of the plurality are enzyme cofactors; substrates for, e.g., biosynthetic or degradative enzymes (e.g., protease substrates), including carbohydrates, nucleoside/nucleotides, amino acids, lipids; vitamins; hormones; nucleic acids; e.g., DNA molecules; or natural products, e.g., bacterial natural products. The library can include any metabolite, precursor, or intermediate of the members listed above. The library can include any combination of members having different characteristics. For example, a library of cofactors can be combined with a library of substrates for biosynthetic or degradative enzymes.

In a preferred embodiment, the library includes at least 10, 10², 10³, 10⁴, 10⁵, 10⁶, 10⁷, or 10⁸ compounds.

In a preferred embodiment, the library includes at least 10, 10², 10³, 10⁴, 10⁵, 10⁶, 10⁷, or 10⁸ of the library members which share a structural or functional characteristic. In other embodiments, the library can include combinations of members sharing structural or functional characteristics. For example, the library can include at least 10, 10², 10³, 10⁴, 10⁵, 10⁶, 10⁷, or 10⁸ of the library members which share a structural or functional characteristic and at least 10, 10², 10³, 10⁴, 10⁵, 10⁶, 10⁷, or 10⁸ of the library members which share a different structural or functional characteristic.

In a preferred embodiment, a combination of two, or more, libraries is tested with a target simultaneously. By way of example, the target can be tested against each (or some) members of a first library, e.g., a cofactor library, and each (or some) members of a second library, e.g., a library of potential substrates. Thus, in the case of two libraries with a first library having 50 members (first₁, first₂, . . . first₅₀) and a second library having 50 members (second₁, second₂, . . . second₅₀ . . . ) the target is tested against all or a plurality of the novel combinations, e.g., against (first₁, second₁), (first₁ second₂) . . . (first₁, second₅₀), and so on.

B. Description of Certain Preferred Embodiments of Thermo-Chemical Sensors and Uses Thereof

The absorption or evolution of heat is a universal property of chemical reactions. Methods described herein link an interaction, e.g., binding, of a test compound or an interactor, e.g., a test ligand or a candidate substrate, with a target (e.g., a target macromolecule, e.g., a target protein or nucleic acid) to a change in heat. The heat output is detected by calorimetry. This allows analysis of the interaction without imposing sharply constraining limitations on the type, range, or specific identity of the activity of the target. By way of example, it allows for the identification of an interactor, e.g., a substrate, for a target having an unknown, poorly characterized, or merely putative or broadly described activity. E.g., where the target is an enzyme, methods of the invention detect a change in heat generated upon conversion of a test substrate(s) into a product(s) or, where the target and interactor are ligand and counter-ligand, upon binding. Some embodiments of the invention require no assumptions about the nature of the target and its interaction with its interactor, e.g., its naturally occurring ligand, substrate, or binding partner. Other methods of the invention incorporate knowledge of or assumptions about the target (and/or interactor) to guide in the choice of potential interactors. E.g., embodiments of the invention use genomic, or other bioinformatic analyses of the target to optimize and/or prioritize the choice of interactors against which to test the target.

Libraries of interaction candidates, e.g., a library of candidate substrate or test ligands as described herein, are also within the scope of the present invention. The methods and compositions, e.g., libraries, of the present application can be used for diagnostic testing, for research purposes, or to screen for agents, e.g., pharmaceutical agents. Agents, e.g., pharmaceutical agents, identified using the methods described herein are also within the scope of the present invention.

In order that the present invention may be more readily understood, certain terms are first defined.

As used herein, a “test compound” also referred to as an “interactor”, is a species which has potential to interact with a target, e.g., a target macromolecule, (e.g., a protein or nucleic acid). The test compound can be a candidate substrate or a test ligand. A test compound can be any agent, including without limitation small organic molecules, metals, peptides, proteins, lipids, glycoproteins, glycolipids, carbohydrates, polysaccharides, nucleic acids (e.g., a nucleoside or nucleotide precursor, a ds or ss DNA molecule, a circular nucleic acid, a super-coiled nucleic acid), an amino acid, (e.g., a D- or L-amino acid or a precursor thereof), a vitamin, a hormone, enzyme substrates, metabolites, transition state analogs, cofactors, natural products (e.g., bacterial natural products) and combination thereof. A library can comprise a plurality of test compounds.

As used herein, a “mixture” or “reaction mixture” can be a complex combination of substances, e.g., impure samples, such as suspensions, natural product extracts, cell homogenates, cell lysates or cell extracts, whole cells, reconstituted systems, biochemical mixtures, biological samples, tissue samples, biological fluids, or colored solutions, which may include more than one test compound.

As used herein, a “candidate substrate” is a substance which gives rise to a different chemical entity when acted on. Exemplary candidate substrates include an enzyme substrate; a metabolite; a cofactor (e.g., a group transfer and energy coupling molecule); a natural product, e.g., a bacterial natural product; a carbohydrate; a polysaccharide; a nucleic acid, e.g., a nucleoside or nucleotide precursor, a double-stranded (ds) or single-stranded (ss) DNA molecule; an amino acid, e.g., a D- or L-amino acid or a precursor thereof; a vitamin; a hormone; a lipid, among others.

As used herein, a “test ligand” is a member of a combinatorial library; is a drug candidate; is from a library of compounds; a library of natural compounds, e.g., fungal products or fermentation products; organic synthesis libraries. In a preferred embodiment the ligand is: a polypeptide which has been expressed from a nucleic acid from a population of nucleic acids, e.g., from a cDNA library, a differentially expressed cDNA library, a genomic library, a library produced by expression profiling, a library which has been enriched for species expressed in a predetermined tissue, a predetermined time of development, or in a predetermined disorder or in the absence of a disorder, a plurality of nucleic acids which have been selected by hybridization, e.g., by hybridization to an ordered two-dimensional array of probes, a library which was produced after the treatment of a cell or organism with a treatment, e.g., a drug.

As used herein, a “library” is a collection substances which can potentially interact with a target. Preferably, the library includes at least one member which is known to interact with a target.

As used herein, a “substrate library” is a collection of compounds for which targets. e.g., proteins, e.g., those of unknown function (also referred to herein as “unknowns”) can be screened against for potential interaction, e.g., enzymatic activity. Interaction, e.g., enzymatic activity will result in a change in heat output which will be detected by the calorimeter.

As used herein, the term “target” refers to any molecule of interest. In a preferred embodiment: the target is a protein or polypeptide, e.g., a naturally occurring protein or fragment thereof; a protein of unknown function; a protein for which the ligand, substrate, or other interacting molecule is not known. The target can be nucleic acid, e.g., a DNA or RNA (e.g., structured RNA, e.g., a ribozyme). Targets include molecules (e.g., peptides, proteins or nucleic acids), having known or unknown structure or function. In a preferred embodiment, the target is a protein without a catalytic activity, with no known catalytic activity, or has a catalytic activity which is difficult to measure. A target can also be a carbohydrate, a polysaccharide, and a glycoprotein, among others.

As used herein, the term “surrogate ligand” refers to an agent that interacts with (e.g., binds to) a target, e.g., a target protein. The surrogate ligand can be naturally associated with the target, or not naturally associated with the target. In a preferred embodiments the surrogate ligand has a KD for the target of at least 10⁻¹, 10⁻², 10⁻⁴, 10⁻⁶, 10⁻⁸, 10⁻¹⁰, 10⁻¹², 10⁻¹⁵, 10⁻²⁰, 10⁻²⁵, 1⁻³⁰M⁻¹. Preferably, the surrogate ligand meets the following criteria: (1) the surrogate ligand exhibits a heterotropic linkage with respect to a test ligand (i.e., it must be displaced upon binding of a test ligand (negative heterotropic linkage), or its binding to a target is enabled with respect to the test ligand (positive heterotropic linkage); and 2) the surrogate ligand in its “free” or displaced form serves as a switch to generate an amplified signal. For example, the displaced surrogate ligand serves as a substrate for an enzyme. Preferably, the surrogate ligand can interact with (e.g., bind to) any surface or internal sequences, or conformational domains of the target. In other embodiments, the surrogate ligand can catalytically alter the target, or alter the functional activity of the target. Examples of natural surrogate ligands include anions, cations, protons, water and other solution phase components are found in association with a target. Examples of non-naturally occurring surrogate ligands include synthetic protein, a peptide and nucleic acid sequences (e.g., a DNA or an RNA molecule).

A surrogate ligand of the invention is not limited to an agent that interacts with (e.g., binds to) a recognized functional region of the target protein, e.g. the active site of an enzyme, the antigen-combining site of an antibody, the hormone-binding site of a receptor, a cofactor-binding site, and the like.

In a preferred embodiment, the surrogate ligand is a nucleic acid molecule (also referred to herein as a “surrogate nucleic acid ligand”). As used herein, the term “nucleic acid molecule” refers to DNA, RNA, single-stranded or double-stranded and any chemical modifications thereof. Exemplary modifications include, but are not limited to, those which provide other chemical groups that incorporate additional charge, polarizability, hydrogen bonding, and electrostatic interaction to the nucleic acid. Such modifications include, but are not limited to, 2′-position sugar modifications, 5-position pyrimidine modifications, 8-position purine modifications, modifications at exocyclic amines, substitutions of 4-thiouridine, substitution of 5-bromo or 5-iodo-uracil, backbone modifications, methylations, base-pairing combinations such as the isobases isocytidine and isoguanidine, as well as 3′ and 5′ modifications such as capping.

As used herein, the term “surrogate nucleic acid ligand” includes a nucleic acid molecule comprising two to forty nucleotides, preferably ten to thirty nucleotides, more preferably, fifteen to twenty-five nucleotides, and most preferably, twenty nucleotides. Accordingly, in preferred embodiments, the surrogate ligand is an oligonucleotide. In one embodiment, the surrogate nucleic acid ligand is identified using the SELEX procedure as described in detail below, and in Gold et at. (1995) Annu. Rev. Biochem. 64:763-797 entitled “Diversity of Oligonucleotide Functions”; US 5,270,163 entitled “Methods for Identifying Nucleic Acid Ligands” issued in Dec. 14, 1993 to Gold et al.; U.S. Pat. No. 5,567,588 entitled “Systemic Evolution of Ligands by Exponential Enrichment Solutions Selex” issued in Oct. 22, 1996 to Gold et al.; U.S. Pat. No. 5,763,177 entitled “Systemic Evolution of Ligands by Exponential Enrichment: Photoselection of Nucleic Acid Ligands and Solutions Selex” issued in Jun. 9, 1998 to Gold et al.; U.S. Pat. No. 5,874,219 entitled “Method for Detecting a Target Compound in a Substance Using a Nucleic Acid Ligand” issued in Feb. 23, 1999 to Drolet, D. et al.; the contents of all of which are hereby expressly incorporated by reference.

As used herein, the term “signal-generating entity” is an entity which interacts with a surrogate ligand in a non-isothermal process, preferably an exothermic process. Preferably, the signal-generating entity amplifies a signal generated by a test ligand. For example, the signal-generating entity may interact with (e.g., binds to) and preferably, modify a surrogate ligand in a manner that gives rise to a signal, e.g., heat output. A typical signal-generating entity is an enzyme which undergoes an exothermic or endothermic reaction with a surrogate ligand.

Preferably, the signal-generating entity interacts more readily with a free surrogate ligand, as opposed to a surrogate ligand bound to a target. In certain embodiments, the signal-generating entity modifies the free (as opposed to target-bound) surrogate ligand by, e.g., forming or breaking a covalent or a non-covalent bond. For example, the modification step may involve cleavage, degradation, phosphorylation, polymerization, or any other event that generates a signal, e.g., a heat signal. The signal-generating entity can be a degradative enzyme (e.g., a nuclease or a protease). Alternative, the signal-generating entity can be a polymerizing enzyme, e.g., a polymerase. For example, in those embodiments, where the free surrogate ligand is a DNA molecule, the signal-generating entity can be a nuclease, such as a staphylococcal nuclease (SNase), Serratia marcescens nuclease (SNase), bovine pancreatic nuclease (DNase I), or human (type IV) nuclease. Alternatively, the signal-generating entity can be a polymerase, e.g., a Tac polymerase. In those embodiments, where the free surrogate ligand is an RNA molecule, the signal-generating entity can be a ribonuclease (e.g., an RNAse). In those embodiments, where the free surrogate ligand is a protein or a peptide, the signal-generating entity can be a protease. Exemplary proteases include, but are not limited to, trypsin, chymotrypsin, V8 protease, elastase, carboxypeptidase, proteinase K, thermolysin, papain and subtilisin. In those embodiments where the surrogate ligand is a metal ion, an enzyme requiring the metal for activation can be used as the signal-generating entity.

In other embodiments, the signal-generating entity can be a solution (e.g., a buffer solution) that amplifies the molecular events which occur when a test ligand binds to a target (e.g., a target protein). For example, many target proteins release or bind a large number of protons when they bind to a test ligand. These release or absorption events are said to be “linkage” events. The linkage process can be amplified by introducing in the solution a buffer molecule with a large heat of ionization, for example, Tris HCl. In yet other embodiments, the signal-generating entity can be a change in phase, or an aggregation or polymerization of material.

As used herein, the interaction of a first molecule with a second can include a change in the association of the two molecules, e.g., an increase or decrease, in the binding of the two molecules or a modification of either or both of the molecules. As used herein modification includes, making or breaking a bond, e.g., a non-covalent or covalent bond. It includes cleavage, degradation, hydrolysis, a change in the level of phosphorylation, labeling, ligation, synthesis, and similar reactions. Modification can include physical changes in phase, changes in aggregation, or polymerization.

In the case of the interaction with a signal-generating entity the modification is not isothermal, and is preferably exothermic.

As used herein, the phrase “analyzing a ligand” can include one or more of: determining if the ligand binds to the target; evaluating the affinity of a test ligand for a target.

Generation of Targets

The targets used in the methods of the present invention can be any molecule of interest. Preferably, the target is a protein or polypeptide (also referred to herein as a “target protein”), e.g., a naturally occurring protein or fragment thereof; a protein of unknown function; a protein for which the ligand, substrate, or other interacting molecule is not known. Exemplary target proteins include, without limitation, receptors, enzymes, oncogene products, tumor suppressor gene products, transcription factors, and infectious proteins (e.g., proteins obtained from an infectious organism, e.g., viral, parasitic, bacterial, and/or fungal proteins). Furthermore, target proteins may comprise wild type proteins, or, alternatively, mutant or variant proteins, including those with altered stability, activity, or other variant properties, or hybrid proteins to which foreign amino acid sequences, e.g. sequences that facilitate purification have been added (e.g., a glutathione S-transferase (GST) moiety).

The target proteins can be either in purified form or in impure form (e.g., as part of a complex mixture of proteins and other compounds as described herein). In certain embodiments, the target protein can be a recombinant protein or a biochemical isolate. For example, the target can be any protein encoded by a gene isolated from a prokaryotic or eukaryotic organism. The isolated gene can be cloned into an expression vector, and introduced into a suitable host cell under conditions which allow expression of the cloned genes by practicing standard molecular biology techniques (Ausubel, F. et al., eds. Current Protocols in Molecular Biology 1999, J. Wiley: New York.; Sambrook, J., Fritsh, E. F., and Maniatis, T. Molecular Cloning: A Laboratory Manual. 2nd, ed., Cold Spring Harbor Laboratory, Cold Spring Harbor Laboratory Press, Cold Spring Harbor, N.Y., 1989). Vectors can be, e.g., plasmids, viral vectors, among others. Preferably, the vectors are modified, e.g., by linking the gene encoding the target protein to appropriate regulatory sequences, such that appropriate expression of the target protein is obtained. Examples of regulatory sequences include promoters, enhancers and other expression control elements (e.g., poly-adenylation signals) (see e.g., Goeddel; (1990) Gene Expression Technology: Methods in Enzymology 185, Academic Press, San Diego, Calif.).

Targets may be obtained from a prokaryotic or a eukaryotic organism, such as microorganisms (e.g., bacteria, viruses, parasites), vertebrate or invertebrate animals (e.g., mammals, e.g., humans).

Exemplary prokaryotic organisms include: Aquifex aeolicus, Pyrococcus horikoshii, Bacillus subtilis, Treponema pallidum, Borrelia burgdorferi, Helicobacter pylori, Archaeoglobus fulgidus, Methanobacterium thermo., Escherichia coli, Mycoplasma pneumoniae, Synechocystis sp. PCC6803, Methanococcus jannaschii, Mycoplasma genitalium, Haemophilus influenzae, Rickettsia prowazekii, Pyrococcus abyssii, Bacillus sp. C-125, Pseudomonas aeruginosa, Ureaplasma urealyticum, Pyrobaculum aerophilum, Pyrococcus furiosus, Mycobacterium tuberculosis H37Rv, Mycobacterium tuberculosis CSU93, Neisseria gonorrhea, Neisseria meningiditis, Streptococcus pyogenes, Borellia burgdorferi, Caulobacter crescentus, Chlorobium tepidum, Deinococcus radiodurans, Enterococcus faecalis, Legionella pneumophila, Mycobacterium avium, Mycobacterium tuberculosis, Methanococcus jannaschii, Neisseria meningitides, Pseudomonas putida, Porphyromonas gingivalis, Salmonella typhimurium, Shewanella putrefaciens, Streptococcus pneumoniae, Thermotoga maritime, Treponema denticola, Thiobacillus ferroxidans, Vibrio cholerae, Clostridium acetobutylicum, Enterococcus faecium, Mycobacterium leprae, Pseudomonas aeruginosa, Staphylococcus aureus, Bacillus sp., Bartonella henselae, Bordetella pertussis, Campylobacter jejuni, Francisella tularensis, Halobacterium salinarium Institute, Listeria monocytogenes, Mycobacterium tuberculosis Sanger, Mycoplasma mycoides, Neisseria meningitidis strain, Streptomyces coelicolor, Rickettsia prowazekii, Sulfolobus solfataricus, Synechocystis sp. PCC6803, Thermoplasma acidophilum, Yersinia pestis, Xylella fastidiosa, Actinobacillus actinomyce, Chlamydia trachomatis, Halobacterium sp. NRC-1, Mycoplasma capricolum, Neisseria gonorrhea, Pseudomonas aeruginosa, Pyrococcus furiosus, Pyrobaculum aerophilum, Rhodobacter capsulatus, Rhodobacter sphaeroides, Streptococcus pyogenes, Ureaplasma urealyticum, Crenarchaeum symbiosum, Pasteurella multocida, Ehrlichia sp. (HGE agent), Haemophilus ducreyn and Streptomyces hygroscopicus.

Exemplary eukaryotic organisms include: Aspergillus nidulans, Candida albicans, Leishmania major, Neurospora crassa, Pneumocystis caribii, Plasmodium falciparum, Saccharomyces cerevisiae, Schizosaccharomyces pombe, Trypanosoma cruzi, Trypanosoma brucei, Tetrahymena sp., Cryptosporidium parvum, Arabidopsis thaliana M, Brugia malayi, Caenorhabditis elegans, Drosophila melanogaster, Shistosoma mansoni, Shistosoma japonicum, and mammals, e.g., humans.

Targets can be produced by an organelle of a eukaryotic organism. For example, the target can be a mitochondrial enzyme. Examples of the organisms for which the genomes of organelles are known include: Chiorarachnion, Guillardia theta, Cyanophora paradoxa, Epifagus virginiana, Euglena gracilis, Guillardia theta, Marchantia polymorpha, Nicotiana tabacum, Odontella sinensis, Oryza sativa, Porphyra purpurea, Pinus thunbergiana, Acanthamoeba castellanii, Allomyces macrogynus, Bos Taurus, Cafeteria roenbergensis, Chrysodidymus synuroideus, Chondrus crispus, Chlamydomonas reinhardtii, Drosophila melanogaster, Drosophila yakuba, Equus asinus, Homo sapiens, Mus musculus, Ochromonas danica, Porphyra purpurea, Prototheca wickerhamii, Reclinomonas Americana, Saccharomyces cerevisiae, Schizosaccharomyces pombe, Tetrahymena pyriformis and Xenopus laevis.

Targets can also be produced by phage, including without limitation, Acholeplasma bacteriophage, Acholeplasma phage/virus, Bacteriophage bIL67, Bacteriophage Cp-1, Bacteriophage G4, Bacteriophage HP1, Bacteriophage IKe, Bacteriophage lambda, Bacteriophage MS2, Bacteriophage PRD1, Bacteriophage PZA, Bacteriophage T4 and Lactococcus bacteriophage C2.

Targets may also be viral proteins. Examples of the viruses that can produce the target include: Abelson murine leukemia virus, Adeno-associated virus 2, Adeno-associated virus 3, African swine fever virus, Alfalfa mosaic virus, Apple chlorotic leaf spot virus, Apple stem grooving virus, Arabis mosaic virus satellite, Arctic ground squirrel hepatitis B virus, Artichoke mottled crinkle virus, Autographa californica nuclear polyhedrosis virus, Avian carcinoma virus, Avian infectious bronchitis virus, Avian leukosis virus, Avian sarcoma virus, BK virus, Baboon endogenous virus, Baboon endogenous virus (BaEV), Bamboo mosaic virus, Barley yellow dwarf virus, Barmah Forest virus, Bean golden mosaic virus, Beet curly top virus, Beet yellows virus, Black beetle virus, Bombyx mori nuclear polyhedrosis virus, Border disease virus, Boma disease virus, Bovine immunodeficiency virus, Bovine leukemia virus, Bovine viral diarrhea virus, Brome mosaic virus, Cacao swollen shoot virus, Caprine arthritis-encephalitis virus, Cardamine chlorotic fleck virus, Carrot mottle virus A, Cassava common mosaic virus, Cassava latent virus, Cassava vein mosaic virus, Cauliflower mosaic virus, Chicken anemia virus, Chloris striate mosaic virus, Citrus tristeza virus, Clover yellow mosaic virus, Coconut foliar decay virus, Commelina yellow mottle virus, Cucumber green mottle mosaic virus, Cucumber mosaic virus, Cucumber necrosis virus, Dengue virus 3, Dengue virus type 1, Dengue virus type 2, Digitaria streak virus, Duck hepatitis B virus, Ebola virus (constructed), Eggplant mosaic virus, Encephalomyocarditis virus, Equine infectious anemia virus, Feline immunodeficiency virus, Foxtail mosaic virus, Friend murine leukemia virus, Friend spleen focus-forming virus, Fujinami sarcoma virus, Ground squirrel hepatitis virus, Hepatitis A virus, Hepatitis B virus, Hepatitis C virus, Hepatitis D virus, Hepatitis E virus, Hepatitis G virus, Hepatitis GB virus B, Heron hepatitis B virus, Hog cholera virus, Human T-cell lymphotropic virus type 1, Human T-cell lymphotropic virus type 2, Human T-cell lymphotropic virus type 1, Human adenovirus type 12, Human adenovirus type 2, Human foamy virus, Human herpesvirus 1, Human herpesvirus 3, Human herpesvirus 4, Human immunodeficiency virus type 1, Human immunodeficiency virus type 2, Human parainfluenza virus 3, Human respiratory syncytial virus, Infectious hematopoietic necrosis virus, Influenza A virus, Influenza B virus, Influenza C virus, JC virus, Japanese encephalitis virus, Jembrana disease virus, Kennedya yellow mosaic virus, Lactate dehydrogenase-elevating virus, Leishmania RNA virus, Leishmania RNA virus 1, Lucerne transient streak virus, Maize streak virus, Maize streak virus, Marburg virus, Mason-Pfizer monkey virus, Measles virus, Melon necrotic spot virus, Mice minute virus, Molluscum contagiosum virus subtype 1, Moloney murine sarcoma virus, Mouse mammary tumor virus, Murine leukemia virus, Murine osteosarcoma virus, Murine sarcoma virus, Mushroom bacilliform virus, Narcissus mosaic virus, Onyong-nyong virus, Odontoglossum ringspot virus, Olive latent virus 1, Ononis yellow mosaic virus, Ovine pulmonary adenocarcinoma virus, Panicum streak virus, Papaya mosaic virus, Papaya ringspot virus, Pea early browning virus, Pea seed-borne mosaic virus, Peanut chlorotic streak virus, Peanut stripe virus, Peanut stunt virus, Pepper huasteco virus, Pepper mottle virus, Plum pox virus, Polyomavirus strain a2, Polyomavirus strain a3, Potato leaf roll virus, Potato mop-top virus, Potato virus A, Potato virus M, Potato virus X, Potato virus Y, Punta Toro virus, Rabbit hemorrhagic disease virus, Rabies virus, Rice tungro spherical virus, Rice yellow mottle virus, Ross River virus, Rous sarcoma virus, Rubella virus, Saccharomyces cerevisiae virus La, Saguaro cactus virus, Satellite tobacco necrosis virus, Sendai virus, Simian foamy virus, Simian immunodeficiericy virus, Simian sarcoma virus, Simian virus 40, Sindbis virus, Sindbis-like virus, Sonchus yellow net virus, Southern bean mosaic virus, Soybean chlorotic mottle virus, Spiroplasma virus, Strawberry vein banding virus, Sulfolobus virus-like particle ssvl, Swine vesicular disease virus, Theiler's encephalomyelitis virus, Tick-borne encephalitis virus, Tobacco etch virus, Tobacco mild green mosaic virus, Tobacco mosaic virus, Tobacco necrosis virus, Tobacco vein mottling virus, Tomato bushy stunt virus, Tomato golden mosaic virus, Tomato leaf curl virus, Tomato yellow leaf curl virus, Turnip vein-clearing virus, Turnip yellow mosaic virus, Vaccinia virus, Variola virus, Venezuelan equine encephalitis virus, Vesicular stomatitis virus, Visna virus, West Nile virus, Woodchuck hepatitis B virus, Woodchuck hepatitis virus, Y73 sarcoma virus, and Yellow fever virus.

Bioinformatic Analysis of Targets

In preferred embodiments, a putative or predicted function is assigned to the target, preferably, prior to testing the target with a test compound. To assign a putative function to the target, e.g., protein, several bioinformatic techniques can be used. The identification of a characteristic shared (or in some cases not shared) by the target and a molecule, e.g., a protein, of known function can allow assignment of the, or an, activity of the molecule, e.g., protein, of known function to the target. Examples of the methods currently used to predict protein functions include: sequence-based searches, fold recognition techniques (including threading algorithms and neural networks), homology modeling, and structure-based analyses.

For example, using FASTA, BLAST and Smith-Watermann algorithms, a pairwise sequence comparison of a given sequence against all those with known function can be carried out (Shpaer, E. G. et al. (1996) Genomics 38(2)). To identify conserved regions of a protein, e.g., sequence motifs that may be predictive of function, two techniques can be used: PROSITE [http ://expasy.hcuge.ch/sprot/prosite.html] and BLOCKS [http://www.blocks.fhcrc.org/blocks/]. PROSITE analysis relies on matching patterns of amino acid residues using a consensus sequence or motif. A second approach, BLOCKS, matched sequences against a full ungapped multiple sequence alignment of the conserved region not just the consensus sequence, and can therefore be highly sensitive at picking out distantly related sequences.

The target amino acid sequence can also be analyzed for the presence or absence of protein folds using the Class, Architecture, Topology (fold family) and Homologous superfamily (CATH) database [http://www.biochem.ucl.ac.uk/bsm/cath]. Currently, more than 670 different types of protein folds are represented in this database. The ability to predict these structural motifs from primary sequences can be improved through the use of threading techniques (i.e. fitting the amino acid sequence of a protein of interest along a known 3-dimensional protein structure) (Bryant, S H et al. (1993) Proteins 16:92-112). Furthermore, neural networks can also be used to predict the fold of proteins (Bohr, H. et al. (1990) FEBS Lett. 261, 43-46).

Additional predictions of the accuracy of the function of a target protein which is homologous to another protein of known function (provided that at least 30-35% of the amino acid sequences are identical) can be obtained by homology modeling. Homology modeling (Johnson, M S et al. (1994) Crit. Rev. Biochem. Mol. Biol. 29:1-68) involves the use of computational algorithms to compare the amino acid sequence of a protein of interest with that of another related protein with known 3-dimensional structure.

Furthermore, structure-based determination of protein function can be used to infer a biological function for a target. For this analysis, the crystal structure of a target protein is determined, and its 3-dimensional structure is then compared with other proteins of known function. If there is a match, a biological function for a target can be predicted based on the known functions of the other protein. This approach was recently used to identify a novel NTPase from Methanococcus jannaschii (Hwang, K Y et al. (1999) Nat. Struct. Biol. 6: 691-696).

Generation of Test Compounds

A test compound (an interactor) is a chemical compound, molecule or complex, which can be tested for its ability to interact with (e.g., bind to) a target, e.g., a target protein. In one embodiment, the test compound is a small organic molecule, e.g., a synthetic or a naturally-occurring non-proteinaceous molecules. The test compound can be designed such that it interacts with a target, or it can be selected from a library of diverse compounds (e.g., a substrate library or a combinatorial library) based on a desired activity, e.g., random drug screening based on a desired activity (e.g., its ability to interact with a target).

Libraries

Method of the invention use libraries as sources of candidate interactors, e.g., agents which are candidates to be tested for the ability to interact with a target. A library can include a plurality of structurally or functionally related members. Library members can, however, be unrelated by structure or function.

A library which includes a plurality of members which are functionally or structurally related can be useful, particularly when the target can be assigned an activity or a putative activity. For example, in the case where the target may be a nuclease, a nuclease substrate library can be tested against the target. A nuclease substrate library can include a range of substrates or putative substrates.

Libraries can be directed to broad target “activities”. Examples of libraries are discussed below. The substrate requirements of newly discovered enzymes can be determined by dividing the substrate library in a systematic manner. This methodology will be referred to herein as substrate profiling.

Cofactor Libraries

A “cofactor” library can include any of: group transfer and energy coupling molecules, coenzyme, e.g., ATP, GTP, TTP, CTP, UTP, NADH, NADPH, NAD, NADP, FAD, FADH, phosphoenolpyruvate, Coenzyme A, lipoamide, 5-adenosylmethionine, Thiamine pyrophosphate, Biotin, tetrahydrofolate, Uridine diphosphate glucose, Cytodine diphosphate diacylglycerol, and all known CoA modifying molecules such as succinyl-CoA. These group of molecules, called, cofactors are involved in vast number of diverse enzymatic reactions. An example of a cofactor library is disclosed and tested in Example 5 below, and includes the following members: ATP, GTP, CTP, TTP, UTP, NADH, NADPH, NAD, NADP, FAD, Flavin, Thiamine Monophosphate Chloride, Pyrodoxal 5′-phosphate, Coenzyme A, and Cocarboxylase.

In a preferred embodiment, the library includes at least 1, 2, 5 or 10 of the members disclosed herein. In many cases a cofactor library will be tested together with another library, for example, a cofactor library can be tested in combination with a carbohydrate library (see Example 5, below).

Carbohydrate Metabolism Libraries

Carbohydrate metabolism libraries, e.g., biosynthesis and/or degradation libraries can be used to screen for carbohydrate modifying enzymes. They can include carbohydrates, e.g., those involved in known biochemical pathways including long, short and single unit carbohydrates and modified carbohydrates from known biochemical pathways such as phosphorylated carbohydrates. A library of this type can include carbohydrates or modified carbohydrates not yet known to be substrates for any enzymes. Examples of the carbohydrates that can be used include: Glucose, Fructose, Arabinose, Xylose, Mannose, Galactose, Lactose, Sucrose, and Ribose. Additional examples of substrates which can be used in the carbohydrate library are provided in the Metabolic Pathway Chart, 1997, 20^{th} edition, from Sigma-Aldrich. In a preferred embodiment, the library includes at least 1, 2, 5 or 10 of the members disclosed herein. The carbohydrate library can be tested in combination with other libraries, e.g., a cofactor library.

An example of a carbohydrate library is disclosed and tested in Example 5 below, and includes the following members: D-glucose, arabinose, sucrose, ribose, lactose, galactose, maltose, and xylose tested.

Purine and Pyrimidine Metabolism Libraries

Purine and pyrimidine metabolism libraries, e.g., biosynthesis and/or degradation libraries can include nucleoside/nucleotide precursors and can be used to screen for enzymes involved in purine and pyrimidine biosynthesis or degradation. Examples of the purine and pyrimidine compounds that can be used include: Glycinamide-ribose-phosphate, Urea, Formyl glycinamide-RP, 5-Aminoimidazol carboxylate-RP, Inosine-P, Formylamido-imidazle-carboxamide-RP. Additional examples of substrates which can be used in the purine and pyrimidine library are provided in the Metabolic Pathway Chart, 1997, 20^{th} edition, from Sigma-Aldrich. In a preferred embodiment, the library includes at least 1, 2, 5 or 10 of the members disclosed herein. The purine and pyrimidine metabolism library can be tested in combination with other libraries, e.g., a cofactor library.

Amino Acid Metabolism Libraries

Amino acid metabolism libraries, e.g., biosynthesis and/or degradation libraries can include amino acids, both D and/or L form, and precursors of the amino acids. It can include peptides with known protease domains to serve as substrates for all the currently known proteases. This library will also contain some non-enzymatic proteins such as BSA to test for proteolytic activity not yet discovered or categorized, allowing the discovery of new classes of proteases. Examples of the amino acids that can be used in the amino acid metabolism library include: Alanine, Aspartate, Cysteine, Histidine, Glycine, and Isoleucine. Additional examples of substrates which can be used in the amino acid metabolism library are provided in the Metabolic Pathway Chart, 1997 20^{th} edition, from Sigma-Aldrich. Examples of a peptide to be used as protease substrates include acetyl-ser-gln-asn-tyr-pro-val-val amide (from Sigma, page 1132, catalogue number A0806, 1999 edition) and Ser-pro-Arg also from Sigma. In a preferred embodiment, the library includes at least 1, 2, 5 or 10 of the members disclosed herein. The amino acid metabolism library can be tested in combination with other libraries, e.g., a cofactor library.

Lipid Metabolism Libraries

Lipid metabolism, e.g., a biosynthesis and/or degradation library, can include fatty acids, fatty acid precursors, steroids and steroid precursors, both those already discovered as substrates for known enzymes as well as fatty acids and steroids not yet discovered or categorized as substrates for enzymes. This library can be used to screen for enzymes involved in fatty acid metabolism. Examples of the substrates that can be used in the lipid metabolism libraries include: cholesterol, desmosterol, Zymosterol, Lanosterol, choline, lecitin, cephalin, linoleate, cardiolipin, and acetylcholine. Additional examples of substrates which can be used in the lipid biosynthesis and degradation library are provided in the Metabolic Pathway Chart, 1997, 20^{th} edition, from Sigma-Aldrich. In a preferred embodiment, the library includes at least 1, 2, 5 or 10 of the members disclosed herein. The lipid metabolism library can be tested in combination with other libraries, e.g., a cofactor library.

Vitamin and Hormone Libraries

This class of library can include vitamins and hormones as well as their metabolic precursors and can be used to screen for enzymes involved in the synthesis, breakdown or modification of hormones of vitamins. Examples of the substrates that can be used in the vitamin and hormone library include: retinoate, metarhodopsin, rhodopsin, vitamin K, opsin, and vitamin E. Additional examples of substrates which will be used in the vitamin and hormone library are given in the Metabolic Pathway Chart, 1997, 20^{th} edition, from Sigma-Aldrich. In a preferred embodiment, the library includes at least 1, 2, 5 or 10 of the members disclosed herein. The vitamin and hormone library can be tested in combination with other libraries, e.g., a cofactor library.

DNA Molecule Libraries

This class of library can be used to screen for DNA modifying enzymes. It can include ds and ss DNA molecules, as well as partially ds DNA molecules, and DNA of random sequence, such as calf thymus DNA. It can include covalently closed circular DNA, both supercoiled and relaxed. These DNA molecules can be obtained from commercial vendors such as Sigma, Amersham, and Biorad. In a preferred embodiment, the library includes at least 1, 2, 5 or 10 of the members disclosed herein. The DNA molecule library can be tested in combination with other libraries, e.g., a cofactor library.

Natural Product Libraries

Natural product libraries, e.g., bacterial natural product library can contain the natural products of an organism, e.g., a bacterium. They can be used to screen for unknown enzymatic activity amongst the unknowns. The substrate requirements of the natural product library can be determined by the deconvolution of the natural products by chromatographic methods. In a preferred embodiment, the library includes at least 1, 2, 5 or 10 of the members disclosed herein. The natural product library can be tested in combination with other libraries, e.g., a cofactor library.

In certain embodiments, the natural product, e.g., the bacterial natural product, is generated in vivo, e.g., by using a mutant organism (e.g., a temperature-sensitive bacterial mutant, or an auxotroph) which accumulates a given metabolite when grown at non-permissive conditions, e.g., at non-permissive temperature, or in the absence of an essential nutrient. The accumulated metabolite can be then purified from the organism prior to testing.

Each of the different substrate libraries can be incubated with the target protein/proteins with the cofactor library at several different pH values and a common mixture of different salts in solution. Any enzymatic activity can be detected as a change in the heat output detected by the calorimeter. This will allow us to immediately categorize the broad type of enzymatic activity the new protein has. The precise substrate requirements can then be determined by dividing the substrate library systematically. Finally the substrate requirements and solution conditions for the newly discovered enzymes can be optimized, and important parameters such as the k_{cat}, k_{M} and/or k_{D} can be determined.

In other embodiments, the test compound can be a member of a combinatorial library. Combinatorial libraries can be synthesized using methods known in the art and as reviewed in, see, e.g., E. M. Gordon et al., J. Med. Chem. (1994) 37:1385-1401; DeWitt, S. H.; Czarnik, A. W. Acc. Chem. Res. (1996) 29:114; Armstrong, R. W.; Combs, A. P.; Tempest, P. A.; Brown, S. D.; Keating, T. A. Acc. Chem. Res. (1996) 29:123; ElIman, J. A. Acc. Chem. Res. (1996) 29:132; Gordon, E. M.; Gallop, M. A.; Patel, D. V. Acc. Chem. Res. (1996) 29:144; Lowe, G. Chem. Soc. Rev. (1995) 309, Blondelle et al. Trends Anal. Chem. (1995) 14:83; Chen et al. J. Am. Chem. Soc. (1994) 116:2661; U.S. Pat. Nos. 5,359,115, 5,362,899, and 5,288,514; PCT Publication Nos. WO92/1 0092, WO93/09668, WO9 1/07087, WO93/20242, WO94/0805 1).

Libraries of test ligands can be prepared according to a variety of methods known in the art. For example, a “split-pool” strategy can be implemented in the following way: beads of a functionalized polymeric support are placed in a plurality of reaction vessels; a variety of polymeric supports suitable for solid-phase peptide synthesis are known, and some are commercially available (for examples, see, e.g., M. Bodansky “Principles of Peptide Synthesis”, 2nd edition, Springer-Verlag, Berlin (1993)). To each aliquot of beads is added a solution of a different activated amino acid, and the reactions are allow to proceed to yield a plurality of immobilized amino acids, one in each reaction vessel. The aliquots of derivatized beads are then washed, “pooled” (i.e., recombined), and the pool of beads is again divided, with each aliquot being placed in a separate reaction vessel. Another activated amino acid is then added to each aliquot of beads. The cycle of synthesis is repeated until a desired length is obtained. The residues added at each synthesis cycle can be randomly selected; alternatively, residues can be selected to provide a “biased” library. It will be appreciated that a wide variety of peptidic, peptidomimetic, or non-peptidic compounds can be readily generated in this way.

In another illustrative synthesis, a “diversomer library” is created by the method of Hobbs DeWitt et al. (Proc. Natl. A cad. Sci. U.S.A. 90:6909 (1993)). Other synthesis methods, including the “tea-bag” technique of Houghten (see, e.g., Houghten et al., Nature 354:84-86 (1991)) can also be used to synthesize libraries of compounds according to the subject invention.

Combinatorial libraries of compounds can be synthesized with “tags” to encode the identity of each member of the library (see, e.g., W. C. Still et al., U.S. Pat. No. 5,565,324 and PCT Publication Nos. WO 94/08051 and WO95/28640). In general, this method features the use of inert, but readily detectable, tags, that are attached to the solid support or to the compounds. When an active compound is detected (e.g., by one of the techniques described above), the identity of the compound is determined by identification of the unique accompanying tag. This tagging method permits the synthesis of large libraries of compounds which can be identified at very low levels. Such a tagging scheme can be useful to identify compounds released from the beads.

In preferred embodiments, the libraries of test ligands contain at least 30 compounds, more preferably at least 100 compounds, and still more preferably at least 500 compounds. In preferred embodiments, the libraries of test ligands contain fewer than 10⁹ compounds, more preferably fewer than 10⁸ compounds, and still more preferably fewer than 10⁷ compounds.

Formats

The methods taught herein can be performed in a number of physical formats. In a preferred embodiment, the measurement is performed in an isothermal titration calorimeter. The calorimeter, e.g., an isothermal titration calorimeter, can be equipped with a flow cell. In a preferred embodiment the target is immobilized in the flow cell. Samples can be introduced into a flow cell from a multi-compartment sample holder, e.g., a multi-well plate such as a microtitre plate, e.g., a 96 well plate. Samples can be pre-mixed in the compartments of the sample holder.

In other preferred embodiments a multi-compartment sample holder, e.g., a multi-well plate such as a microtitre plate, e.g., a 96 well plate, in which each compartment includes a thermopyle, and each is a calorimetric cell can be used. Channels for fluid delivery to the compartments can be included.

In a preferred embodiment a method can be performed on a microchip, in which the appropriate wells channels, and other components have been formed, e.g., by etching or deposition. Fluids could be pumped or moved by electrokinetic methods.

In methods described herein the reaction mixture can include a single target or multiple targets. Similarly, one, or more, ligand can be added to a reaction mixture. It may be useful to multiplex one or both of these elements in order, e.g., to screen large numbers of species. E.g., where a large number of ligands are to be evaluated, the initial group of candidates can be pooled, and if a pool shows a promising result, members of the pool evaluated. Likewise in methods for evaluating substrates, candidates can be pooled.

For large scale screening, calorimetry can be combined with a high-throughput screening format. For the purposes of high-throughput screening, the experimental conditions described above are adjusted to achieve a threshold proportion of test ligands identified as “positive” compounds or ligands from among the total compounds screened. Preferably, this threshold is set according to two criteria. First, the number of positive compounds should be manageable in practical terms. Second, the number of positive compounds should reflect ligands with an appreciable affinity towards the target protein. A preferred threshold is achieved when 0.1% to 1% of the total test ligands are shown to be ligands of a given target.

ITCs are commercially available and are used routinely by skilled artisans. See e.g., U.S. Pat. No. 5,873,763 issued to Plotnikov, V. V. on Sep. 29, 1998; Indyk et al. (1998) Meth. Enzymol. 295:350-364; Brandts et al. (1990) American Laboratory 30-41. The ITC is a twin-cell differential device. It operates at a fixed temperature, while the liquid in the sample is continuously stirred. This instrument measures the heat that is evolved or absorbed as a result of the binding of the test ligand to the target.

In other embodiments, a differential scanning microcalorimeter (DSC) can be used to detect the heat output. DSCs are commercially available and are used routinely by skilled artisans. See e.g., U.S. Pat. No. 5,873,763 issued to Plotnikov, V. V.; and Freire (1995) Meth. Mol. Bio. 40:191-218. The differential scanning microcalorimeter automatically raises or lowers the temperature at a given rate while monitoring the temperature differential between cells. From the temperature differential information, small differences in the heat capacities between the sample cell and the reference cell can be determined and attributed to the test substance.

In a preferred embodiment: the reaction mixture is not transparent; the reaction mixture is colored; the reaction mixture is turbid; the reaction mixture contains a substance which interferes with fluorescent or calorimetric detection; the reaction mixture is not a pure solution, e.g., it contains products other than the target. In a preferred embodiment the reaction mixture contains: a substance which interferes with radioactive analysis; a substance which interferes with spectrophotometric analysis, e.g., NMR analysis. In a preferred embodiment a complex mixtures of substances, e.g., an impure sample, such as a suspension, natural product extract, cell extract, biochemical mixture, or colored solution, which may include more than one test compounds, is tested.

Surrogate Ligand-Based Methods

Identification of Surrogate Nucleic Acid Ligands

A surrogate ligand can be a nucleic acid (e.g., an oligonucleotide). The SELEX procedure can be used to identify a surrogate ligand. Using the SELEX procedure (Gold et al. (1995) supra), a large number of random sequence oligonucleotides can be tested for their ability to bind with high affinity to a target, e.g., a target protein. The larger the library of nucleotides, the greater the chance of finding at least one sequence which binds to the target with a dissociation constant in the picomolar to nanomolar range. Preferably, the ligand is about 20 nucleotides in length, as longer oligonucleotides will presumably only bind using a fraction of their length, leaving some residues vulnerable to degradation or processing by a signal-generating entity, even while the ligand is bound to the target. For example, in the case of a surrogate nucleic acid ligand, longer oligonucleotide sequence may lead to background hydrolysis when a DNase is used.

In one embodiment, the target used in the methods of the invention is a protein (e.g., a target protein). Initially, the target protein can be identified and purified, using standard biochemical techniques such as HPLC and ion-exchange or size-exclusion chromatography. Preferably, a highly purified sample of the target protein is obtained. Then, the SELEX method is employed to identify a single-stranded oligonucleotide (DNA) ligand which binds to the protein with high (>nM) affinity. The first step in this process entails the generation of a random oligonucleotide library of 10¹⁴-10¹⁵ single-stranded DNA sequences which having the structure from the 5′ to the 3′ end shown below:

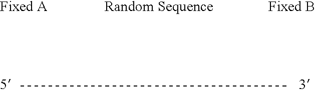

where Fixed A and B refer to constant sequences present at the 5′ and the 3′ ends of each member of the library. These constant sequences flank a random sequence and allow transcription and subsequent pool amplification after each round of the SELEX process (see Tuerk &Gold (1990) Science 249:505-510). Preferably, the random sequences should not range beyond 20 nucleotides in length, (as larger ligands may only bind to the target using a central span of their residues, thus leaving their termini exposed to the activity of the DNase even while they are still bound to the target protein).

Next, the library is mixed with the target protein and then partitioned by passage through a nitrocellulose membrane. Those DNA sequences bound to the filter by the protein are then eluted and amplified with the polymerase chain reaction (PCR) for subsequent transcription of the (now-modified) library for a second round of SELEX. The process is repeated until a ligand which binds with the desired affinity is obtained.

It has been shown that each round of the method produces on average a 10-fold enrichment of the high affinity ligands (Schneider et al. (1992). J. Mol. Biol. 228:862-869), and a range of between 10 and 20 rounds of SELEX are usually necessary to identify a ligand which binds with a Kd in the nanomolar range (Gold et al., 1995, supra). In the initial rounds, it is advisable to use a fairly high amount of protein to insure the retention of all the high-affinity ligands when far more abundant low-affinity ligands are present in the library. The selectivity of the SELEX process can be increased by using lower amounts of the target (e.g., target protein) in the later rounds, when the high-affinity ligands have been enriched enough to survive the competitive binding situation. The process has been tried with over 30 proteins and in almost all cases oligonucleotide ligands were found which bound with greater than nM affinity (Gold et al., 1995, supra).

Selection of the Signal-Generating Entity

Once a surrogate ligand has been identified, a suitable signal-generating entity is chosen which has high specific activity against the surrogate ligand. Preferably, the signal-generating entity interacts (e.g., binds) more readily with a free surrogate ligand, as opposed to a surrogate ligand bound to a target. Preferably, such interaction amplifies a signal, e.g., generates a heat signal. In certain embodiments, the signal-generating entity modifies the free surrogate ligand by, e.g., forming or breaking a covalent or a non-covalent bond. For example, the modification step may involve cleavage, degradation, phosphorylation, polymerization, or any other event that generates a signal, e.g., a heat signal. The signal-generating entity can be a degradative enzyme (e.g., a nuclease or a protease). Alternative, the signal-generating entity can be a polymerizing enzyme, e.g., a polymerase.

The signal-generating entity can be immobilized, e.g., attached to a solid support, or crosslinked. For example, in those embodiments where the signal-generating entity is an enzyme. the enzyme can be cross-linked to form a crystalline enzyme.

For example, in those embodiments where the free surrogate ligand is a DNA molecule, the signal-generating entity can be a nuclease. Exemplary nucleases that can be used include without limitation staphylococcal nucleases (SNase), Serratia marcescens nucleases (SNase), bovine pancreatic nucleases (DNase I), or human (type IV) nucleases. As the activity can vary over many orders of magnitude, even for a single nuclease and a variety of oligonucleotide substrates, the optimal DNase may be chosen. Optimal solution conditions may also be chosen, e.g., pH, temperature, and solvent conditions. The activity of a particular DNase for a specific surrogate ligand can be assayed using methods described in Friedhoff et al. (1996) Eur. J. Biochem. 241:572-580 and Friedhoff et al. (1999) FEBS Lett. 443:209-214. Several exemplary DNases are listed in the table below, along with their activities against particular substrates. The table is by no means complete, and it is not intended to limit the scope of the present invention.

TABLE 1
Steady-state parameters for the cleavage of various
nucleic acid substrates by several nucleases.

| Enzyme | Substrate | k_{cat/}Kₘ(s⁻¹ μM⁻¹) | Reference |
|---|---|---|---|
| Snase^{a} | salmon sperm DNA | 15 | Poole et al. (1991) Biochemistry |
| | | | 30:3621-3627 |
| Dnase 1^{b} | calf thymus DNA | 1.6 | Doherty et al. (1995) |
| | | | J. Mol.Biol. |
| | | | 251(3):366-77 |
| SM6^{c} | salmon testis DNA | 54 | Friedhoffet al.(1996) supra |
| BNase^{d} | salmon DNA | 7.2 | Brown & Ho (1987) |
| | | | Eur. JBiochem. |
| | | | 168:357-364 |

^{a}staphylococcal nuclease
^{b}bovine pancreatic deoxyribonuclease
^{c}Serratia Marcescens endonuclease
^{d}barley nuclease

The signal-generating entity can also be a polymerase, e.g., a Tac polymerase. In those embodiments, where the free surrogate ligand is an RNA molecule, the signal-generating entity can be a ribonuclease (e.g., an RNAse).

In other embodiments where the free surrogate ligand (as opposed to target-bound) is a protein or a peptide, the signal-generating entity can be a protease. Proteases useful in practicing the present invention include without limitation trypsin, chymotrypsin, VS protease, elastase, carboxypeptidase, proteinase K, thermolysin and subtilisin (all of which can be obtained from Sigma Chemical Co., St. Louis, Mo.). The most important criterion in selecting a protease or proteases for use in practicing the present invention is that the protease(s) must be capable of digesting the particular target protein under the chosen incubation conditions. To avoid “false positive” results caused by test ligands that directly inhibit the protease, more than one protease, particularly proteases with different enzymatic mechanisms of action, can be used simultaneously or in parallel assays. In addition, cofactors that are required for the activity of the protease(s) are provided in excess, to avoid false positive results due to test ligands that may sequester these factors.

Calorimetric Ligand Screening Using a Surrogate Nucleic Acid

The description below exemplifies the use of a surrogate nucleic acid ligand and a target protein. The experimental conditions described herein can be easily extended to the use of other surrogate ligands (e.g., protein ligands) and targets by the skilled artisan. Briefly, once a surrogate ligand (e.g., a surrogate nucleic acid ligand) is identified which binds to the target protein with a suitable dissociation constant, a solution of the target protein and the surrogate nucleic acid ligand is prepared in a 1:1 ratio (concentrations approximately 10 mM) and allowed to equilibrate inside the microcalorimetry cell for several minutes, along with a much smaller concentration of a signal-generating entity. For example, a specific deoxyribonuclease (DNAse), at a concentration of approximately 1 nM, which has been chosen for its high activity against the surrogate nucleic acid ligand identified in the SELEX process. One assumption in the present invention is that while the surrogate nucleic acid ligand is bound to the target protein, it is prevented from undergoing as rapid a degradation by the DNAse as the free surrogate nucleic acid ligand.

More specifically, the target protein, surrogate nucleic acid ligand, and specific nuclease are then combined together in solution to form a reaction mixture. The target protein and surrogate nucleic acid ligand can both be present at approximately 1 μM, while the nuclease is present at approximately 1 nM. The total sample volume is approximately 1 ml. The sample is incubated in the microcalorimetry cell (e.g., the cell of an isothermal titration calorimeter). The twin cells are housed in an insulated container. The container is cooled, so heat energy is required to maintain the cells and their contents at the experimental temperature. The two cells are kept at thermal equilibrium with each other. A small aliquot (5-25 μl) of a test ligand that potentially binds to the target protein is then added to the sample cell. If the test ligand binds to the target protein with significant affinity (relative to the oligonucleotide), it will release some fraction of the surrogate ligand into solution, depending on the magnitudes of the respective binding constants. This newly-liberated surrogate ligand will then begin to be hydrolyzed by the nuclease present in the solution, thus generating a heat output (power output) much larger than that produced by the initial competitive binding of the test compound. The heat output can be recorded for approximately 1 minute. The total heat output for a given trial can be related to (e.g., is proportional to) the ratio of the affinities of the surrogate ligand and the test ligand for the target protein.

In other embodiments, the conformational change of a target upon test ligand binding can be measured. Protein targets and structured RNA targets (e.g., ribozymes) have one common feature: they undergo a conformational change to a less compact form upon addition of a denaturant to the solution, or when heat is added by an increase in temperature. The conformational change that a protein or RNA undergoes is accompanied by a large change in heat that may conveniently be detected by a calorimeter. If a test ligand binds to the more compact form of the target protein or RNA, then it will inhibit the conformational change thereby allowing for a difference in heat output to be detected as compared to a control solution with no test ligand. A detailed description of this detection is provided in Examples 3 and 4.

The methods can also be used to analyze (e.g., identify) agents that bind to a target where the target is present on the surface of a cell, e.g., a bacterial cell wall component. Thus, the methods can be used to identify agents that interact with (e.g., bind to) cell surface molecules.

The interaction among the target and the surrogate ligand, test ligand, and/or substrate can occur in vitro (e.g., in a cell-free system), or in vivo (e.g., in a cell, e.g., a prokaryotic or an eukaryotic cell). For example, this interaction can be tested by adding these compounds to cells, e.g., living cells, placed inside of a calorimeter.

In order that the invention described herein may be more fully understood, the following examples are set forth. It should be understood that these examples are set forth for illustrative purposes only and are not to be construed as limiting this invention in any manner.

### EXAMPLE 1

### Screening for Compounds Capable of Binding to a Target Protein

First, a single-stranded oligonucleotide of DNA is identified which binds to the target protein of interest with high affinity. For the purpose of this example, the ligand is assumed to be 20 residues long and has a kd=1 nM. The protein and the oligonucleotide ligand are mixed together, each with a concentration of 10 μM. A minute amount (1 nM) of a deoxyribonuclease known to have high activity against the ligand is added (total volume of the solution 500 μl). The binding reaction between the target protein (P) and the ligand (L) can be written as follows:

Kd

PL=P+L and rearranged in the form: Kd=[P][L]/[PL] (equation 1)

Knowing the concentration of the protein-ligand complex [PL] allows the calculation of the amount of free protein and free ligand in the solution, from the expressions

[Pₜ]=[P]+[PL] (equation 2)

and

[Lₜ]=[L]+[PL] (equation 3)

Substituting equations 2 and 3 into equation 1 and solving for [PL] gives the quadratic expression:

[PL]²−(L_{T}+PTₜ+K_{d})[PL]+P_{T}L_{T}=0

which can be solved for [PL]. In this case, where [P_{T}] and [L_{T}] are both 10 μM and Kd=1 nM, [PL] is equal to 0.99 μM, which means that 1% of the ligand is free in solution (from equation 3). This small fraction will begin to be hydrolyzed once the protein-ligand solution is combined with the DNase (giving some baseline heat output: 70 μcal/sec×10 sec⁻¹=700 μcal/sec).

After the initial solution of target protein, ligand, and nuclease is allowed to equilibrate in the sample cell, a test compound is added, at a final concentration of 10 μM. Assuming for the purposes of this example that it has an identical Kd for the target as the original oligonucleotide, the test compound will eventually compete off one-half of the DNA, leaving it to be degraded by the DNase. The total amount of DNA in the cell is approximately 5×10⁻⁹ moles. If half of this is hydrolyzed completely, this means that (20)(2.5×10⁻⁹)=5×10⁻⁸ moles of phosphodiester bonds are cleaved, giving a total heat output of (70 kcal/mol)(5×10⁻⁸ mol)(10 sec⁻¹)=35,000 μcal/sec. This is a factor of 50 larger than the baseline heat output from the original 1% free ligand.

### EXAMPLE 2

### Calorimetric Detection of Enzymatic Turnover and Inhibition

The measure of activity of an enzymatic reaction is directly proportional to the differential power output in the calorimetric cell resulting from catalyzed conversion of substrate to product. The detection signal (power) for an enzyme reaction, as monitored by a calorimeter, is equal to the substrate turnover per second times the heat of the reaction, as given by the following equation:

ΔP=ΔH×V=ΔH [−dS/dt]

where S is the substrate concentration and V is the enzymatic rate.

To measure changes in enzyme activity that are as small as 5%, the change in power output of the calorimeter need be no greater than 0.5 μcal/sec. This power change would result from substrate turnover of 5×10⁻¹¹ moles/sec assuming a typical heat of reaction of 10 kcal/mol of substrate. Since turnover numbers for enzymes are in the range from 10 to 10,000 per second, one would only require as little as picomoles or femtomoles of enzyme in the calorimeter to perform each drug screening assay.

To optimize the calorimetric assay to ensure maximum signal change, it is helpful to consider the following. A simple form of the equation relating velocity of an enzyme to the concentration of substrate and inhibitor is:

V=Vmax{1+Kₘ/S[1+I/Ki]}⁻¹

where S and I are the substrate and inhibitor concentrations, respectively, Kᵢ is the inhibitor dissociation constant and Km is the Michaelis-Menton parameter for substrate. Kₘ values vary predominantly in the range from 10⁻¹ to 10⁻⁶ M. The enzyme activity is most sensitive to changes in small concentrations of inhibitor when the substrate concentration equals the Kₘ. Addition of an inhibitor at a concentration equal to K₁, to a solution of enzyme with substrate concentration Km reduces the velocity of the enzyme (and hence the power output) by 34%.

### EXAMPLE 3

### Detection of a Test Ligand by Measuring the Heat of Conformation Change of a Target Using an Isothermal Calorimeter

After purifying the target of interest (e.g., a target protein or structured RNA), a solution of a buffered solution is denaturant such as Guanidine hydrochloride or urea is introduced into the calorimetric reaction cell. Then a syringe is filled with protein at a specified concentration (−100 μM). Injections of approximately 10 μL (containing 1 nanomole of target) are introduced into the calorimetric cell. For a typical protein, 100 kcal/mol is absorbed when the protein undergoes its conformational change to a less compact state, so that 100 microcalories can be detected, after subtracting out any heat effect due to the dilution of the denaturant. The heat effect due to the dilution of the denaturant will be small since 10 μL titrant are being added to over 1000 μL of solution. However, the actual value for this dilution effect can be determined by adding 10 μL of buffer without protein into the solution of denaturant.

In order to detect if a test ligand binds to the protein, one can repeat the above experiment, modifying it so that the test ligand is introduced into the denaturant solution and the protein solution at equal concentrations. If the test ligand binds to the compact state of the protein, then a smaller amount of the protein will convert to the less compact state upon being injected into the calorimeter, leading to a change in the overall heat output. The injection process could easily be automated by coupling a flow injection system to a titerplate, so that each sample is introduced into the same larger volume of denaturant.

### EXAMPLE 4

### Detection of a Test Ligand By Measuring the Heat of Conformation Change of a Target Using a Differential Scanning Calorimeter

A buffered solution containing a purified target (e.g., a target protein of interest) is introduced into the differential scanning calorimeter reaction cell. Heat is added by increasing the temperature of the solution. When the protein undergoes its conformational change to a less compact form, heat is released. The apparent specific heat capacity curve is integrated to obtain the apparent specific heat output due to the conformational transition. The experiment is repeated with a fresh solution of protein to which some test ligand has been added. If the test ligand binds to the protein, the apparent specific heat output obtained by integrating the apparent specific heat capacity curve between the same two temperature points as the previous experiment, will be less. This difference serves as a convenient signal to indicate which ligands bind to the protein.

### EXAMPLE 5

### Hexokinase Substrate Profiling Experiment

This example shows the monitoring of the rate of heat production resulting from the phosphorylation of glucose by hexokinase using isothermal titration calorimetry (ITC).

Materials and Methods:

Hexokinase type F-300 from Bakers yeast was purchased from Sigma and used without further purification. ATP and the carbohydrate and coenzyme kits used for substrate profiling were also obtained from Sigma. All reagents were suspended in a solution containing 100 mM HEPES (pH 8.0), 10 mM MgCl2, 10 mM KCl and 1 mM in ATP (reaction buffer). In order to create multiple combinations of carbohydrate and coenzyme mixtures (see flow chart below), stock solutions for each individual component were made up to 2 and 1 mM respectively, such that final dilution's with the reaction buffer yielded 1 mM carbohydrate and 0.5 mM coenzyme. Hexokinase was prepared as a 50-unit/ml stock solution in reaction buffer.

Reaction enthalpies reflecting enzyme turnover were obtained from thermograms collected with a VP-ITC microcalorimeter (MicroCal Inc., Northampton, Mass.). The VP-ITC instrument directly measures the heat evolved or absorbed in liquid samples as a result of injecting precise amounts of reactants into a thermally equilibrated reaction cell. The reaction cell volume is approximately 1.7 mls and is enclosed with an identical reference cell in an adiabatic inner shield inside an adiabatic outer shield. Once the instrument has been completely assembled with a spinning syringe it is brought to the desired experimental temperature. As the cells reach thermal equilibrium, temperature differences between the reference cell and the sample cell are measured. Calibration of the differential power (DP signal) between the reference cell and the sample cell is obtained electrically by administering a known quantity of power through a resistive heating element on the cell. An injection which results in the chemical evolution (exothermic) or absorption of heat (endothermic) within the sample cell causes a negative change in the DP signal for an exothermic reaction and a positive change in the DP signal for an endothermic reaction. Since these chemical changes result in heats that deflect the initial (electrically equilibrated) DP signal away from equilibrium the instrument's DP feedback readministers power back into the cell compensating for these changes. Thus, the DP signal display has units of power (μcal/sec) and the time integral of the peak yields a measurement of thermal energy, ΔH.

The reaction conditions for the hexokinase substrate profiling were as follows: the sample cell was filled with 2 mls of the substrate/coenzyme solution described above. The assay was initiated by injecting 6 μL of 50-Unit/ml hexokinase solution into the sample cell. The temperature during each calorimetric assay was held constant throughout each experiment at 25°. Under the conditions of the experiments represented here, heats of dilution and mixing (as measured by the heat evolved in the absence of substrate) were less than 5% of the total heat measured for the enzymatic reaction (see FIGS. 3B). Throughout the enzymatic reaction the rate of heat generated was monitored continuously as shown in FIGS. 3A-3B. The heat flow reaches a maximum soon after the addition of enzyme to the reaction cell and then decays to the baseline as the level of substrate is depleted (see FIG. 3A). As can be seen from FIGS. 3A and 3B, the amount of heat generated directly reflects whether substrate is present or not.

The actual experimental protocol is summarized by the flow chart shown in FIG. 4. For example, in the first experiment hexokinase is injected into the complete carbohydrate library. The carbohydrate library used contained: D-glucose, arabinose, sucrose, ribose, lactose, galactose, maltose, and xylose tested in the presence of a cofactor library, which included, ATP, GTP, CTP, TTP, UTP, NADH, NADPH, NAD, NADP, FAD, Flavin, Thiamine Monophosphate Chloride, Pyrodoxal 5′-phosphate, Coenzyme A, and Cocarboxylase.

A heat signal is observed similar to that shown in FIG. 3A. This signal is indicative of enzyme turnover and hence the presence of substrate. For the next set of experiments, the complete carbohydrate library is divided into two—one with substrate (Carbohydrate library 2A) the other without (Carbohydrate library 2B) (FIG. 4). The experiment is repeated as before and this time only one sample generates a heat signal, that of Carbohydrate library 2A. Since no detectable heat signal is observed for Carbohydrate library 2B (see FIG. 4) this collection is discarded. Next, we divided the Carbohydrate library 2A into two. The experiment is repeated as before, once again only one sample generates a heat signal. The sample containing substrate is divided in half again and the whole process is repeated, until by the process of elimination we arrive at a single component sample that contains the proper enzyme substrate—glucose.

All of the above-cited references and publications are hereby incorporated by reference.

Although the present invention has been described above in terms of specific embodiments, it is anticipated that other uses, alterations and modifications thereof will become apparent to those skilled in the art given the benefit of this disclosure. It is intended that the following claims be read as covering such alterations and modifications as fall within the true spirit and scope of the invention. It is also intended that the articles “a” and “an”, as used in the claims, include both the singular and plural forms of the nouns that the articles modify.

What is claimed is:

1. A method comprising:
(a) assaying a target by
(i) assigning a putative function to the target;
(ii) providing a library of interaction candidates;
(iii) providing a reaction mixture comprising the target;
(iv) contacting the target with the one or more members of the library of interaction candidates and measuring a value corresponding to a resultant change in heat output for at least one selected member of the library of interaction candidates;
(b) launching drug discovery employing the target comprising determining at least one pharmacological property of the at least one selected member of the library of interaction candidates.

2. The method of claim 1 further comprising processing the target prior to assaying the targets.

3. The method of claim 1 further comprising characterizing the target prior to assaying the targets.

4. The method of claim 1 further comprising identifying and selecting a target prior to assaying the target

5. The method of claim 4 wherein identifying and selecting a target comprises
(a) associating a target with a disease state; and
(b) selecting the associated target.

6. The method of claim 4 where identifying and selecting a target comprises
(a) identifying a disease state;
(b) identifying a target associated with the disease state; and
(c) selecting the identified target.

7. The method of claim 2 wherein processing the target comprises isolating and purifying the target.

8. The method of claim 3 wherein characterizing the target comprises determining one or more physical properties of the target.

9. The method of claim 8 wherein the physical properties of the target are determined using at least one technique selected from the group consisting of NMR, EPR, fluorescence, phosphorescence, light scattering, circular dichroism, UV-Visible absorption, infrared spectroscopy, and calorimetry.

10. The method of claim 1 further comprising repeating steps (c)-(f) with a second library member or one or more members of a second substrate library.

11. The method of claim 1 wherein the target is a protein or an enzyme.

12. The method of claim 1 further comprising ranking the target.

13. The method of claim 12 wherein targets are ranked according to protein function, enzymatic activity, or enzymatic turnover.

14. The method of claim 1 wherein a target that binds to a member of the library of interaction candidates is selected for launching drug discovery.

15. The method of claim 1 wherein launching drug discovery further comprises
(a) synthesizing chemical libraries;
(b) assaying the chemical libraries using a selected target by
(i) assigning a putative function to the selected target;
(ii) providing a library of interaction candidates;
(iii) providing a reaction mixture which includes the selected target;
(iv) contacting the selected target with the one or more members of a substrate library;
(v) evaluating a change in heat output of the reaction mixture; and
(vi) optionally comparing the value for heat change obtained with a predetermined value; and
(c) selecting members of the chemical libraries that bind to the target.

16. The method of claim 15 further comprising testing the selected members of the chemical libraries in vivo or in vitro for therapeutic activity.

17. The method of claim 15 wherein the synthesized chemical libraries are analogs or derivatives of members of the substrate library that bind to the target.

18. The method of claim 1 wherein the one or members of the substrate libraries include native substrates, substrate analogs, substrate derivatives, transition state analogs, enzymatic products, enzymatic product analogs, and enzymatic product derivatives.

19. The method of claim 15 further comprising administering a pharmaceutically effective amount of one or more members of the chemical libraries to a mammal.

20. The method of claim 19 further comprising monitoring a disease state in the presence of the administered pharmaceutically effective amount of the one or more members of the chemical libraries.

21. A method of drug discovery for rapid identification of therapeutics comprising:
(a) identifying and selecting a protein target;
(b) assaying the protein target by:
(1) assigning a putative function to the protein target;
(2) providing a library of interaction candidates;
(3) providing a reaction mixture which includes the protein target;
(4) contacting the target with one or more members of the library of interaction candidates;
(5) evaluating a change in heat output of the reaction mixture;
(6) optionally comparing the value for heat change obtained with a predetermined value;
(c) launching drug discovery using at least one member of the library of interaction candidates that elicits a change in heat output.

22. The method of claim 21 wherein the one or more protein targets are chosen that bind to a member of a substrate library.

23. The method of claim 21 further comprising repeating steps (b)(1)-(b)(6) with a second library member or one or more members of a second substrate library.
